(19) United States
(12) Patent Application Publication
(10) Pub. No.: US 2025/0099405 A1
(43) Pub. Date: Mar. 27, 2025

(54) COMPOSITIONS, METHODS, AND DEVELOPMENT OF ARID4B INHIBITORS

(71) Applicants: University of Kentucky Research Foundation, Lexington, KY (US); The George Washington University, Washington, DC (US)

(72) Inventors: Samuel Ofori, Bronx, NY (US); Samuel Gorman Awuah, Lexington, KY (US); Ray-Chang Wu, Washington, DC (US); Mei-Yi Wu, Washington, DC (US)

(73) Assignees: University of Kentucky Research Foundation, Lexington, KY (US); The George Washington University, Washington, DC (US)

(21) Appl. No.: 18/729,967

(22) PCT Filed: Jan. 18, 2023
(86) PCT No.: PCT/US2023/060841

§ 371 Date: Jul. 18, 2024

Related U.S. Application Data
(60) Provisional application No. 63/300,455, filed on Jan. 18, 2022.

(51) Int. Cl. A61K 31/166; A61K 31/167; A61K 31/357; A61K 31/381; A61K 31/495; A61K 31/5375; C07D 295/13; C07D 295/15; C07D 319/18

(52) U.S. Cl. CPC A61K 31/166; A61K 31/167; A61K 31/357; A61K 31/381; A61K 31/495; A61K 31/5375; C07D 295/13; C07D 295/15; C07D 319/18

(57) ABSTRACT

The present disclosure generally relates to classes of compounds that bind the chromo-barrel domain of AT-rich interactive domain 4B (ARID4B). In some aspects, the compounds are of the Formula (II) as described herein, or a diamine composite thereof as set forth in Formula III herein. In some aspects, the compound is selected from compounds 1a, 1b, 1c, 1d, 1e, 2a, 2b, 2c, 2d, 2e, 2f, 2g, 2h, 2i, 2j, (as described herein) or combinations thereof. The present disclosure further considers administration of the compounds to target ARID4B in cells and for the treatment of cancerous breast tissue cells.

### CROSS-REFERENCE TO RELATED APPLICATIONS

This application claims priority to U.S. Provisional Patent Application 63/300,455, filed Jan. 18, 2022, the content of which is hereby incorporated by reference in its entirety.

### GOVERNMENT SUPPORT

The present disclosure was supported by a grant from the National Institutes for Health, Center for Pharmaceutical Research and Innovation (Grant No. P20 GM130456) and the National Cancer Institute (Grant No. R01CA258421-01). The Government may have rights to the invention.

### TECHNICAL FIELD

The present disclosure generally relates to classes of compounds that bind the chromo-barrel domain of AT-rich interactive domain 4B (ARID4B).

### BACKGROUND

Estrogen receptor alpha (ERα) plays a major role in the development and progression of breast cancer. ERα is activated upon binding of 17-β-estradiol (E2) to the ligand binding domain (LBD), resulting in the conformational change and recruitment of crucial coactivators for transcriptional activation of estrogen response element (ERE)-containing target genes. More than 70% of breast cancers are ERα+ and are treatable with endocrine therapies that inhibit estrogen biosynthesis or ERα activity. However, not all patients with ERα+ cancer respond to endocrine therapy and nearly all ERα+ metastatic cancers that initially respond to endocrine therapy will eventually become endocrine therapy-resistant, hormone-independent cancers. There is an urgent need for new and more effective therapies.

AT-rich interactive domain 4B (ARID4B), also known as retinoblastoma binding protein 1-like 1 (RBP1L1), is a member of the ARID family and a vital component of the SIN3A/HDAC1 chromatin remodeling complex. It has also been implicated in breast cancer progression and metastasis, as well as prostate cancer. The ARID4B protein possesses three key domains: the ARID domain for putative DNA binding activity, the chromobarrel domain, and the tudor domain. The latter two bind methylated histones and play critical roles as molecular adaptors in the assembly of the chromatin remodeling complexes. These complexes are associated with endocrine resistance in breast cancer, and likewise ARID4B is highly expressed in human breast cancers. Compelling evidence has shown that ARID4B is a therapeutic target, as applied to endocrine resistance in breast cancers. Targeting the chromobarrel or tudor domain of ARID4B perturbs the chromatin remodeling complexes, in turn effectively disrupting its role in breast cancer progression and endocrine resistance, providing an alternative means of eliminating breast cancer.

### BRIEF DESCRIPTION OF THE DRAWINGS

FIG. 1 depicts an in silico model of ARD150 docked into the chromobarrel domain of ARID4B.

FIG. 2 depicts the structure of ARD150, highlighting the head and tail.

FIG. 3A depicts the Structure-in-silico Activity Relationship (SiAR) used for the design of novel analogs of ARD150. FIG. 3B depicts general structures of Class I & II analogs and FIG. 3C depicts general “head” structures which are permuted against “tails” to synthesize target analogs.

FIG. 4 depicts general synthetic scheme for the synthesis of Class I compounds.

FIG. 5 depicts general synthetic scheme for synthesis of Class II compounds. Spacer=null or —OCH₂—; X=null, —O—, or —S—S—; R1=null or —CH₃; R₂=—H or —CH₃; R₃=—C₆H₅—OC₆H₅CH₂— or —CH₃.

FIG. 6 depicts structures of synthesized analogs of ARD150.

FIG. 7 shows genomic alterations and expression of ARID4B in breast cancers. A shows genomic alterations of ARID4B in breast cancers (cBioPortal). B shows elevated ARID4B mRNA levels in breast carcinoma compared to normal breast (Oncomine). C shows representative images of IHC staining of ARID4B using breast tumor arrays. D shows expression of ARID4B protein was elevated in tumors compared to normal/benign breast tissues. FIG. 7E shows the levels of ARID4B were higher in Grade III tumors compared with Grades I and II. **P<0.01, ***P<0.001.

FIG. 8 shows Elevated ARID4B expression is associated with unfavorable clinical outcomes in ERα+ breast cancers. A and B show Kaplan-Meier survival analyses showed that high ARID4B expression is associated with reduced recurrence-free survival in ERα+ breast cancer. No correlation with ERα-cancers was found. C shows Kaplan-Meier survival analysis showed that elevated ARID4B expression is associated with decreased recurrence-free survival (left), decreased distant metastasis-free survival (middle), and decreased overall survival (right) in patients with ERα+ breast cancer who received systemic endocrine therapy.

FIG. 9 shows ARID4B is a new coactivator for ERα and is involved in constitutive activation of ERα mutant receptors. A shows MCF7 cells transfected with SiControl or SiARID4B were used for RNA-seq and GSEA. ERα pathway is among the top ten pathways affected by knockdown of ARID4B. Results are from 3 biological duplicate from each group. Graph displays category scores as −log₁₀(P value). B shows co-IP assays were performed using MCF7, T47D, and ZR75-1 cells grown in phenol red-free medium with 10% charcoal-dextran stripped FBS for 4 days and treated with or without E2 for 2 hours prior to Co-IP. IP using anti-ARID4B was followed by Western blots to detect co-precipitated ARID4B and ERα. C shows 293T cells transfected with the indicated plasmids were used for Co-IP by anti-HA, followed by immunoblot with indicated antibodies. Enhanced ligand-independent interaction between ARID4B and ERα Y537S and D538G mutants vs. wild-type ERα receptor was detected (compare lanes 1 and 3, 7 and 8). D shows reporter gene assays using ERE-Luc co-transfection of ARID4B significantly increased ligand-independent activity of the ER□ Y537S and D538G mutants vs. wild-type ERα. FE shows knockdown of ARID4B by siRNAs inhibited E2-induced and constitutive expression of GREB1, PGR, and CCND1 in MCF7 and MCF7 Y537S mutant cells. Similar results were observed in ER□+ T47D and ZR75-1cells (not shown). Data are means±SEM from three experiments performed in triplicate. *P<0.05, **P<0.01, ***P<0.001.

FIG. 10 shows Mammary gland-specific knockout of Arid4b compromised tumorigenesis driven by Erbb2. A shows ARID4B expression in mammary gland of control, Arid4b^{MG-/-}, Erbb2^{MGOE}, and Erbb2^{MGOE}Arid4b^{MG-/-} mice at 2 months old, analyzed by Western blot. B shows tumor-free rate of all four genotypes, using littermates and sisters, was monitored for 60 wks. C shows summary of mammary grand tumorigenesis in Erbb2^{MGOE} (n=28) and Erbb2^{MGOE}Arid4b^{-/-} mice (n=23). D shows average number of tumors per animal.

FIG. 11 shows ARID4B KO clones generated in E2-dependent ER□+ MCF7, T47D (A), and in E2-independent MCF7 Y537S and LCC9 cells (B). C shows knockout of ARID4B inhibited cell proliferation in MCF7 and LCC9 cells. Similar results were observed in T47D and MCF7 Y537S cells (not shown). *P<0.05, **P<0.01, ***P<0.001.

FIG. 12 shows mammary gland-specific ARID4B overexpression. (A) shows the targeting vector contains two ROSA26 genomic sequences for gene targeting (5′ and 3′ arms), a diphtheria toxin A fragment gene (DTA), and a mini-gene consisting of a CAGGS promoter, a loxP-STOP-loxP (LSL) cassette, and human ARID4B cDNA. The mini-gene was inserted into the ROSA26 locus by homologous recombination between the two ROSA26 genomic sequences (5′ and 3′ arms) to generate the knock-in allele. Cre recombinase driven by the MMTV promoter (MMTV-cre) excises the “STOP” cassette, allowing ARID4B overexpression. Overexpression of ARID4B in ARID4B^{MG+/OE} mice was confirmed by qRT-PCR using TaqMan probe specific for human ARID4B (B), and by IHC using ARID4B antibody (C).

FIG. 13 shows in (A) shows a schematic of ARID4B domains along with depicted deletions. (B) shows colony formation assays using MCF7 ARID4B KO transfected with the indicated plasmids carrying specific deletions within ARID4B. (C) shows immunoblotting showed comparable expression of full-length and domain deletion ARID4B mutants.

FIG. 14 shows rational design of chemical inhibitors of ARID4B. (A) shows molecular model of full-length ARID4B and the docked small molecule inhibitor (green sticks/spheres) (B) shows expanded view of compound, ARD150 (magenta sticks) bound in the cavity of the chromo domain of ARID4B. Hydrogen bonds are shown as dashed lines. (C) shows chemical structure of ARD150. (D) shows chemical structures of other synthesized scaffolds, RWR18, RWR10, and ARD153 from in silico/ITC studies. (E) shows predicted ligand interactions formed between ARD150 and the chromodomain site residues, hydrogen and hydrophobic or polar interactions shown as dashed lines.

FIG. 15 shows target engagement by ARID4B inhibitors. (A) shows cellular thermal shift assay (CETSA) showed that treatment of MCF7 with ARD150 (10 μM) for 2 h resulted in a shift of ARID4B aggregation temperatures from 49-52° C. to 52-55° C., indicating binding of ARD150 to ARID4B. (B-C) show cells expressing HA-ARID4B (C) or HA-ARID4BIC (D) treated or not with ARD150 (10 μM) for 2 h were used for CESTA. Treatment with ARD150 resulted in a shift of aggregation temperatures of HA-ARID4B (B), whereas no shift was observed for HA-ARID4B□C (C). (D-E) show cells expressing HA-ARID4B (D) or Flag-ARID4A (E) treated or not with RWR-18 (10 μM) for 2 h were used for CESTA. Treatment with RWR-18 resulted in a shift of aggregation temperatures of HA-ARID4B (D), whereas no shift was observed for Flag-ARID4BA. Data are means±SEM from three experiments. *P<0.05, **P<0.01, ns, not significant.

FIG. 16 shows the binding constants (K_{d}), reaction stoichiometry (n), enthalpy (ΔH) and entropy (ΔS) of ARD150 (A) and RWR18 (B) to ARID4B chromodomain were determined by ITC.

FIG. 17 shows ARID4B KO cells generated in MCF7 were used to determine the EC50 of ARID inhibitors ARD150 (A), RWR-18 (B). Similar results were obtained from T47D and MCF7 Y537S but not shown. (C) shows non-tumorigenic MCF10A, which expressed lower ARID4B compared to breast cancer cells (top panel) is insensitive to ARD150 and RWR-18.

FIG. 18 shows RWR-18 suppressed ERα activation. The mRNA levels of GREB1, PGR, ARID4B, and MYD88 from MCF7 (A) and MCF7 Y537S cells (B) pre-treated with RWR-18 (0, 5 and 10 μM, 1 h) followed by +E2 treatment (10 nM, 4 h) were analyzed by qRT-PCR. Data are means±SEM from three biological replicate. ***, P<0.001. ns, not significant.

FIG. 19 shows body weight of mice that received two injections of vehicle or ARD150 at the indicated doses were measured for 14 days (A). Representative HE images of liver and spleen from mice treated with vehicle or ARD150. Images from vehicle and the highest doses of ARD150 (125 mg/kg) were shown (B). (C) shows the plasma concentrations of RWR-18 were determined by LC-MS for up to 24 h following incubation of RWR-18 with mouse blood plasma. Three biological replicates for each time point.

FIG. 20 shows ARID4B binds to trimethylated H3K4. (A) shows wild-type ARID4B and domain deletion mutants were expressed in Sf9 cells, purified by Ni-NTA affinity resin and stained by Coomassie blue. (B) shows histone peptide binding assays (left) showed that wild-type ARID4B binds to trimethylated H3K4 (top right panel). Dot blots showed the input of biotinylated histone peptides (bottom right panel). (C) shows histone peptide binding assays showed that deletion of Tudor and chromo domains impaired binding to trimethylated H3K4 by ARID4B.

FIG. 21 shows ChIP and 3C-qPCR revealed mechanism for ARID4B's function in ERα signaling. (A-B) show genomic organization of PGR and GREB1. The orange bars indicated the ARID4B binding regions within the promoter of each gene identified by ARID4B ChIP-seq. ERα-bound enhancers were indicated by bars (GSE100328). Independent ChIP-qPCR confirmed recruitment of ARID4B to the promoters of PGR (A, right panel) and GREB1 (B, right panel). (C) shows ARID4B mediates looping of PGR promoter and ERα-bound enhancers is determined by 3C-qPCR. ARID4B-binding site on the PGR promoter and two ERα-binding sites (enhancers 1 and 2) are marked by boxes. Primer sets used in 3C-qPCR analysis to detect looping between the enhancer and promoter are indicated (top panel, arrows). 3C-qPCR analysis shows E2-induced looping between the promoter and two enhancers of PGR in MCF7 cells. On the other hand, looping is abolished in ARID4B KO cells. 3C-qPCR of the MYOD1 promoter serves as a control. The levels of qPCR products between the promoter and two flanking enhancers on PGR were normalized against that of MYOD1 promoter in each sample. In each experiment, the normalized level of the qPCR product from one of the control samples was set as 1. Data are means±SEM from three experiments performed in triplicate. *P<0.05, **P<0.01.

FIG. 22 shows ARID4B is important for recruitment of ERα to the enhancers. (A) shows recruitment of ERα to the enhancers of PGR gene in MCF7 cells with or without knockout of ARID4B was compared by ChIP assays using ERα antibody. Knockout of ARID4B reduced ERα recruitment to the enhancers induced by E2. (B) shows acetylation of H3K27 on PGR gene in MCF7 cells with or without knockout of ARID4B was compared by ChIP assays using anti-H3K27Ac antibody Knockout of ARID4B reduced the acetylation of H3K27 on the promoter and enhancers on PGR induced by E2. Normal IgG and MYOD1 gene were used as negative controls for the ChIP. In each experiment, the normalized level of the qPCR product from one of the control samples was set as 1. Data are means±SEM from three experiments performed in triplicate. *P<0.05, **P<0.01, ***P<0.001. Similar results were observed for GREB1 gene (not shown).

FIG. 23 shows RWR-18 inhibits ARID4B binding to H3K4me3. His-ARID4B was pre-incubated with RWR-18 as indicated followed by histone peptide binding assays.

FIG. 24 shows that RWR 18 inhibits tumor growth. The effects of RWR18 on tumor growth was determined using MCF7 xenograft model. When tumor in SCID mice reached 150-200 mm³, mice were randomized to receive vehicle or RWR18 (25 mg/kg) once every two days for 24 days. The graphs showed tumor growth in mice receiving vehicle (A-E) or RWR18 (G-K). L shows are representative images of excised tumors from mice injected with vehicle (top) or RWR18 (bottom). Bar, 5 mm.

FIG. 24 depicts that RWR18 inhibited tumor growth. The effects of RWR18 on tumor growth was determined using MCF7 xenograft model. When tumor in SCID mice reaches 150-200 mm³, mice were randomized to receive vehicle or RWR18 (25 mg/kg) once every two days for 24 days. The graphs showed tumor growth in mice receiving vehicle (A-E) or RWR18 (G-K).

FIG. 25 depicts excised tumors from mice treated with vehicle (top) or RXR18 (bottom). Bottom graph depicts that the number of apoptotic cells increases in RWR18 treated cells over vehicle. The bottom panel of FIG. 25 shows the effects observed with ARD153 in WT MCF7 cells and in ARID4B KO cells.

### DESCRIPTION

The present disclosure generally relates to classes of compounds that bind the chromo-barrel domain of AT-rich interactive domain 4B (ARID4B). Large-scale genomic analyses of TCGA and other breast cancer datasets showed that ARID4B is amplified in breast cancer (up to 22%). Genome-wide transcriptome analysis and IHC confirmed that expression of ARID4B is elevated in breast cancer cohorts. Clinical data clearly showed that elevated ARID4B expression is associated with high grade tumors and unfavorable clinical outcomes in patients with ERα+ breast cancer treated with systemic endocrine therapies. It has also been identified that ARID4B interacts with and activates not only the wild-type ERα, but also the constitutively active ERα Y537S and D538G mutants in a ligand-independent manner. Results from a novel mammary gland-specific Arid4b knockout mouse model show that knockout of Arid4b inhibisd mammary gland tumorigenesis, suggesting a causative role of Arid4b in breast cancer. Collectively, these data provide compelling evidence to support a critical role for ARID4B in the tumorigenesis and therapy resistance of breast cancer, and a strong rationale for developing inhibitors for pharmacological inhibition of ARID4B.

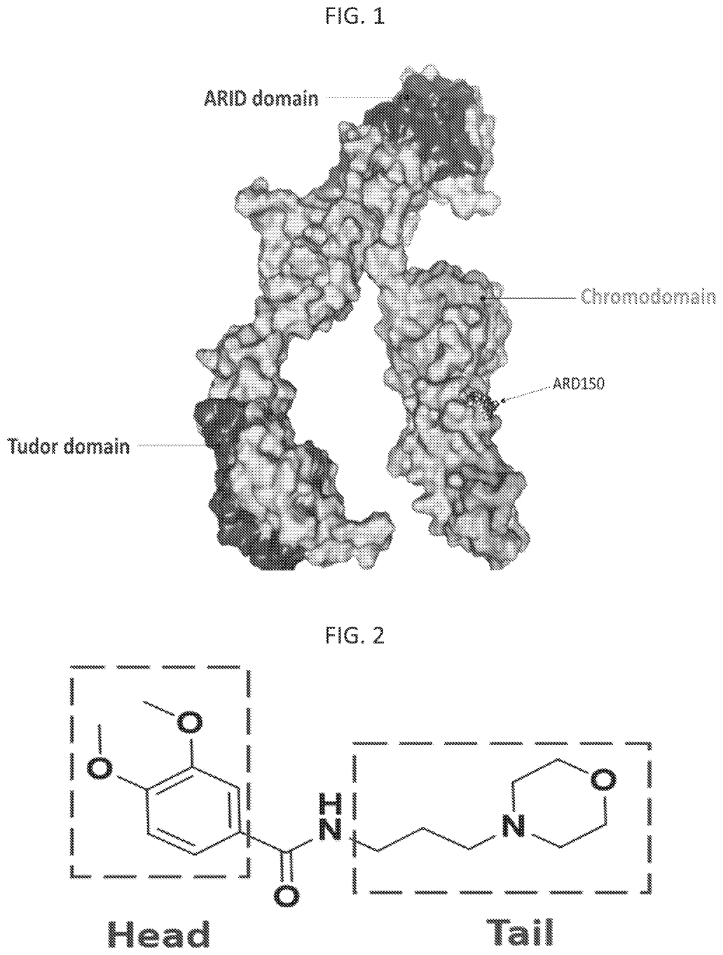

The rational development of a small molecule inhibitor (SMI) of a given protein-protein pathway involves a multidisciplinary approach: computational studies, chemical synthesis, characterization of compounds, and biological characterization. ARID4B lacks a defined crystal structure which requires a heavier emphasis on in silico studies to develop SMIs for this pathway. This multidisciplinary approach is used herein to rationally design small molecule inhibitors of ARID4B protein, ARD150, which targets the chromodomain as seen in FIG. 1. Initial biological characterization through cell-based assays and in vitro/in vivo studies have shown that ARD150 targets ARID4B and is tolerable in mice at extremely high concentrations (125 mg/kg). However, it exhibits poor potency (in the micromolar range). Thus, even though ARD150 is a first-in-class inhibitor, further optimization is necessary to improve its characteristics as a theoretical treatment option for endocrine-resistant breast cancer.

In some aspects, the present disclosure concerns derivatives from the base compound ARD150, the structure of which is set forth in Formula I:

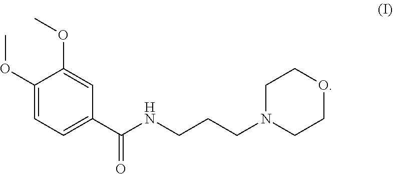 [SMILES: COc1ccc(C(=O)NCCCN2CCOCC2)cc1OC]

As set forth in FIG. 1, ARD150 binds to the chromobarrel domain of ARID4B. It is an aspect of the present disclosure to provide structural derivatives of AD150 that provide improved binding and/or specificity for ARID 4B. In some aspects, the present disclosure concerns structural analogues and/or derivative from ARD150 that increase potency and selectivity to ARID4B and/or the chromobarrel therein. In some aspects, the present disclosure concerns modifications to one or two substructures within ARD150. As depicted in FIG. 2, there are two sub-regions within the structure of ARD150 for modification, the head and tail domains. In some aspects, the present disclosure concerns modifications to the 3,4-alkoxy substituted aromatic head region of ARD150. In some aspects, the present disclosure concerns modifications to the solubilizing tail region of ARD150. In some aspects, the present disclosure concerns modifications to the head and tail regions of ARD150. In some aspects, this disclosure seeks to expand this class of small molecule inhibitors of ARID4B to improve their potency. ARD150 consists of two critical structural frameworks, a 3,4-alkoxy substituted aromatic head and a solubilizing tail, as shown in FIG. 2.

In developing more potent analogs of ARD150, two structural modifications were considered: modification of the aromatic core and the solubilizing tail. The aromatic core modification seeks to enhance pi-pi interaction with amino acid residues in the chromobarrel domain of the ARID4B. Modification of the solubilizing tail seeks to increase the number of hydrogen bond donors on the tail, thereby increasing the number of hydrogen bond interactions in the protein pocket of the chromobarrel domain.

In some aspects, the derivatives may include modifications to the benzene ring of the head group identified in FIG. 2. In some aspects, the benzene ring is an α, β, or γ bond away from the amide bond. In some aspects, the amide functionality can be replaced with oxime, carbamate, or triazole. In some aspects, the benzene ring may feature a substitution at a meta and/or para position. In some aspects, benzyloxy substitutions at the para position of the benzene ring may be included. In some aspects, ortho- and/or halo substitutions are avoided. In some aspects, methyl, ethyl, and/or propyl substitutions on the benzene ring are avoided. In some aspects, a methoxy substitution on the benzene ring is included. In some aspects, substitution of an arylether on the benzene ring is included.

In some aspects, the head group is selected from one of Formula Ia, Ib, Ic, and Id:

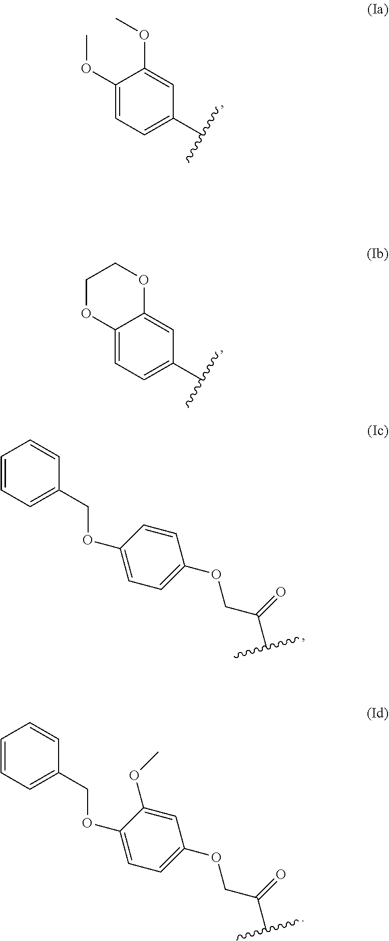

In some aspects, the present disclosure concerns a first class or grouping of ARD150 derivatives that comprise spacer between the benzene ring and the amide of the head group as identified in FIG. 2. In some aspects, the ARD150 structural derivative may comprise a compound as set forth in Formula II:

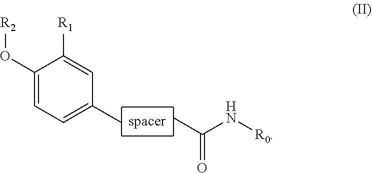

With respect to Formula II, in some aspects, the spacer is null or —OCH₂. In some aspects, R₁ is null, H, or CH₃. In some aspects, R₂ is H or CH₃. In some aspects, R₀ is selected from:

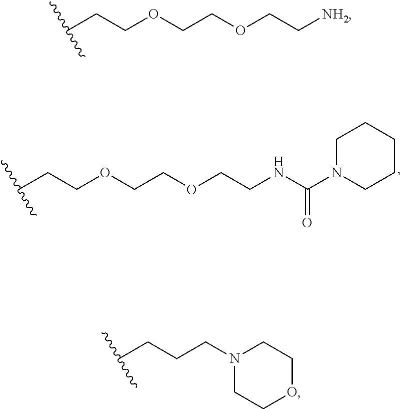 [SMILES: *CCOCCOCCN ; *CCOCCOCCNC(=O)N1CCCCC1 ; "," ; *CCCN1CCOCC1]

or a piperazine with the nitrogen of the depicted amide.

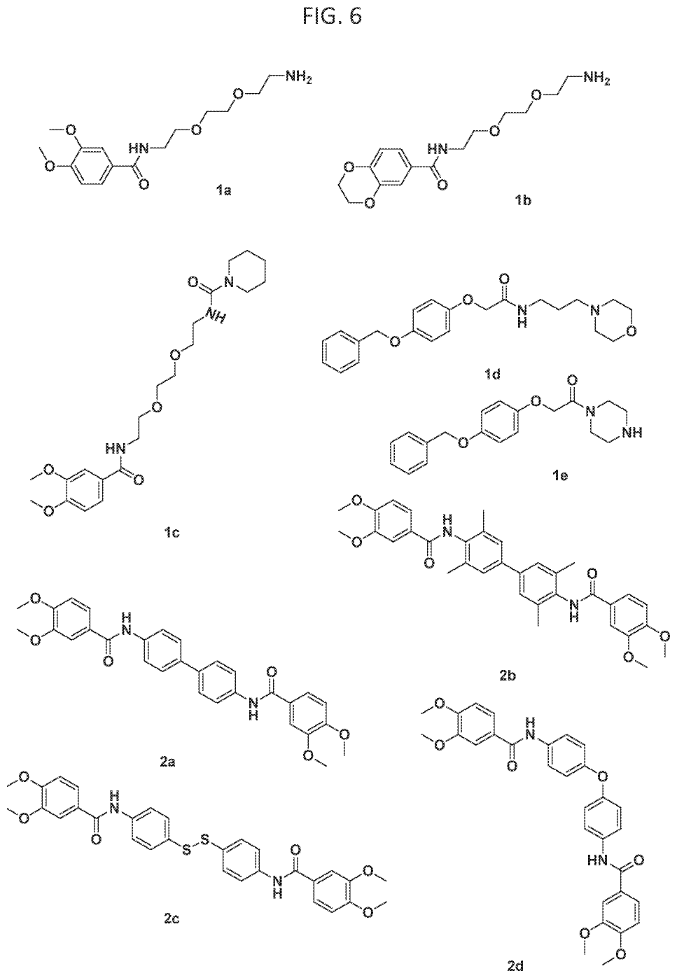

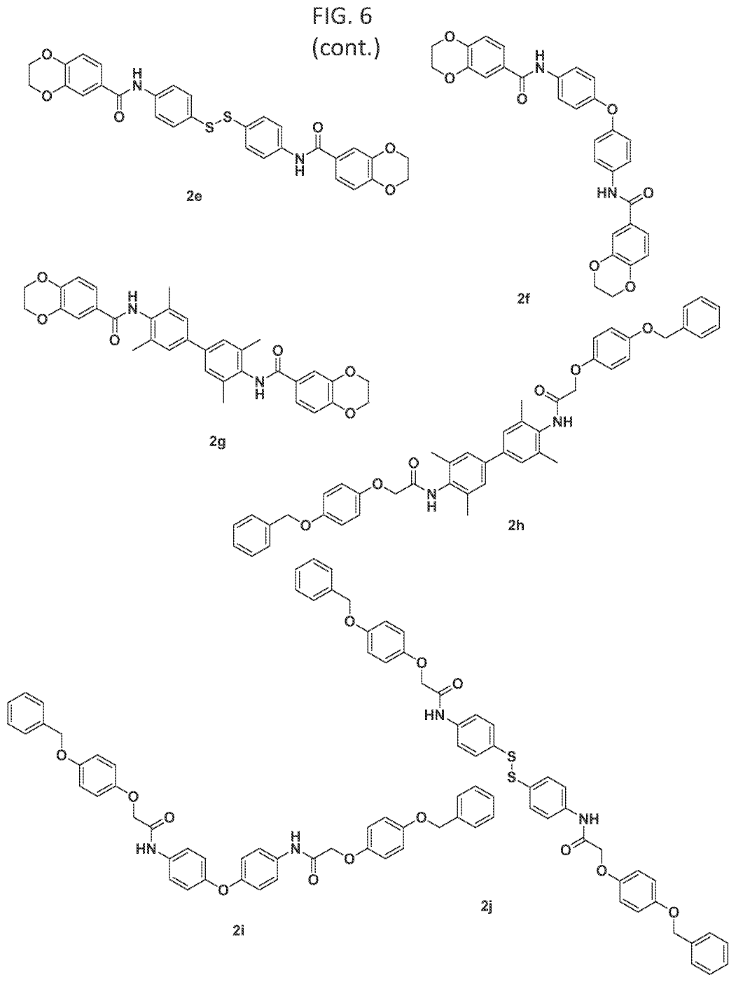

In some aspects, the compound of the first class or grouping of compounds (Class I) is selected from compounds 1a-1e as set forth in FIG. 6.

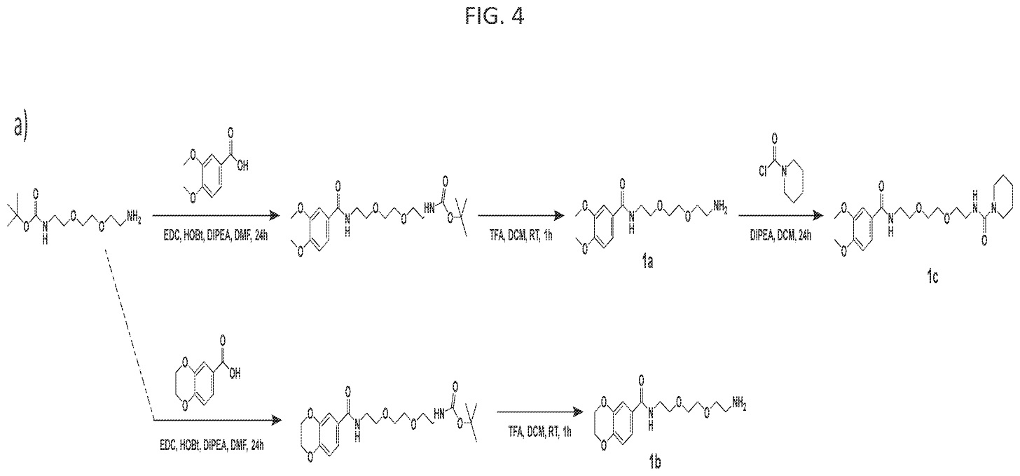

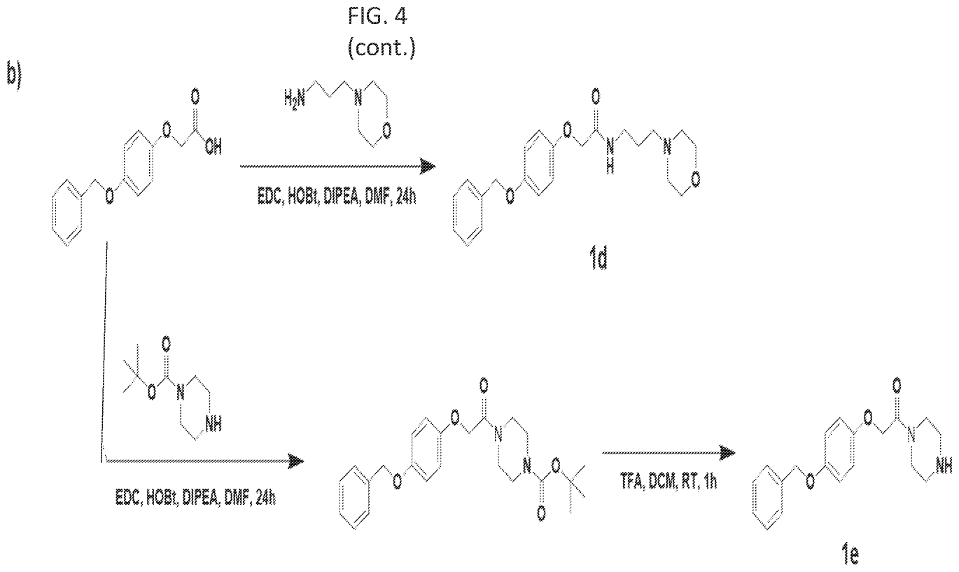

In some aspects, Class I analogs are characterized by modifications of the “tail” section (e.g., 1a-1e in FIGS. 4A and 4B and FIG. 6).

In some aspects, the compound may have the structure as follows:

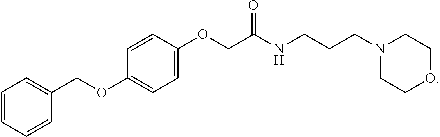 [SMILES: O=C(COc1ccc(OCc2ccccc2)cc1)NCCCN1CCOCC1]

In some aspects, the compound is 1d as set forth in FIG. 6. In some aspects, compound 1d is also referred to as RWR18 or RWR-18 herein.

In some aspects, the compound may have the structure as follows:

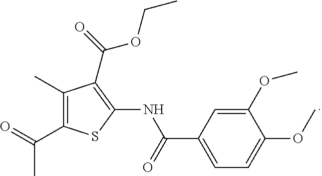 [SMILES: CCOC(=O)c1c(NC(=O)c2ccc(OC)c(OC)c2)sc(C(C)=O)c1C ; "."]

In some aspects, the compound is referred to as ARD153 herein.

In some aspects, the composition of Formula II bookends a diamine with a basic structure as set forth in Formula III:

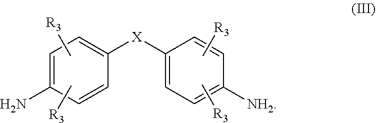

In some aspects, each R3 is independently null, C₆H₅OC₆H₅CH₂, or CH₃. In some aspects, X is null, O, or S—S. In certain aspects, the diamine is selected from the following structures:

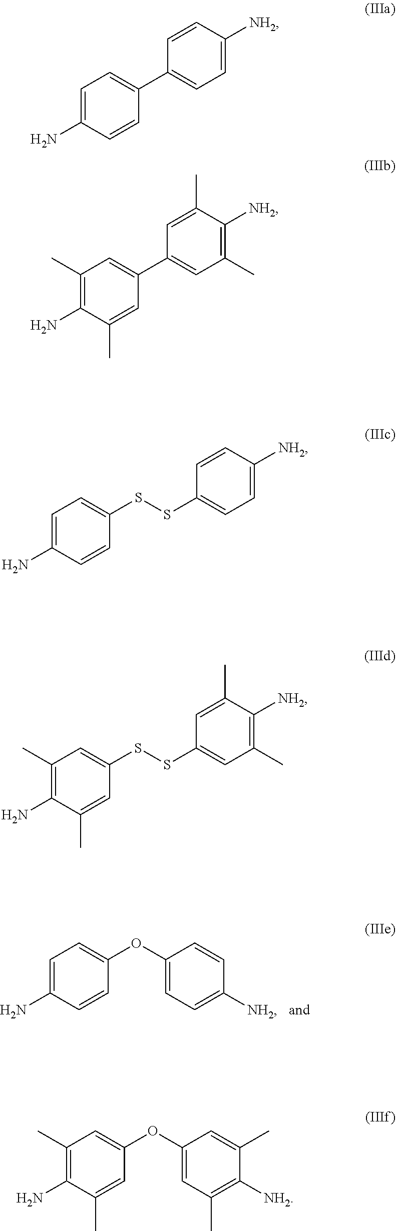

In some aspects, the bookended or symmetrical compounds are set forth in Formula IV:

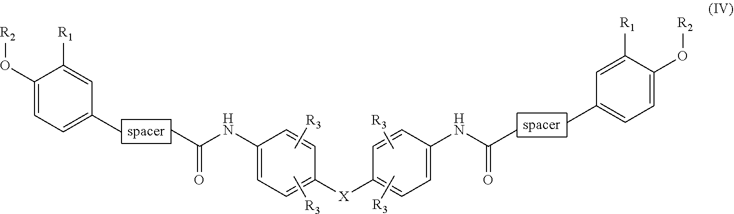

With respect to Forumla IV, in some aspects, each spacer is independently null or OCH₂. In some aspects, each R₁ is independently null, H, or CH₃. In some aspects, each R₂ is independently H or CH₃. In some aspects, each R3 is independently null, C₆H₅OC₆H₅CH₂, or CH₃. In some aspects, X is null, O, or S—S. The overall scheme for the compounds of the second class or grouping (Class II) is set forth below:

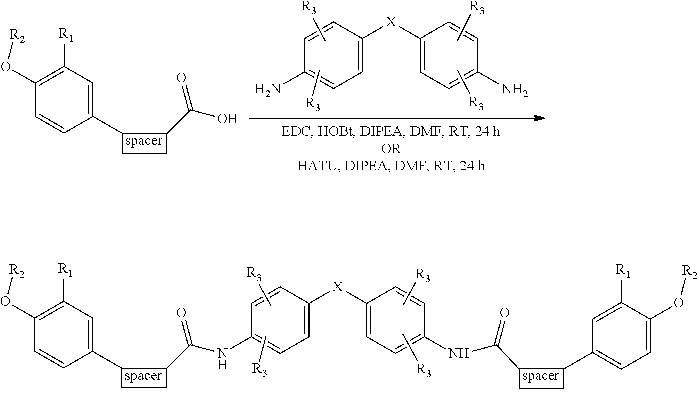

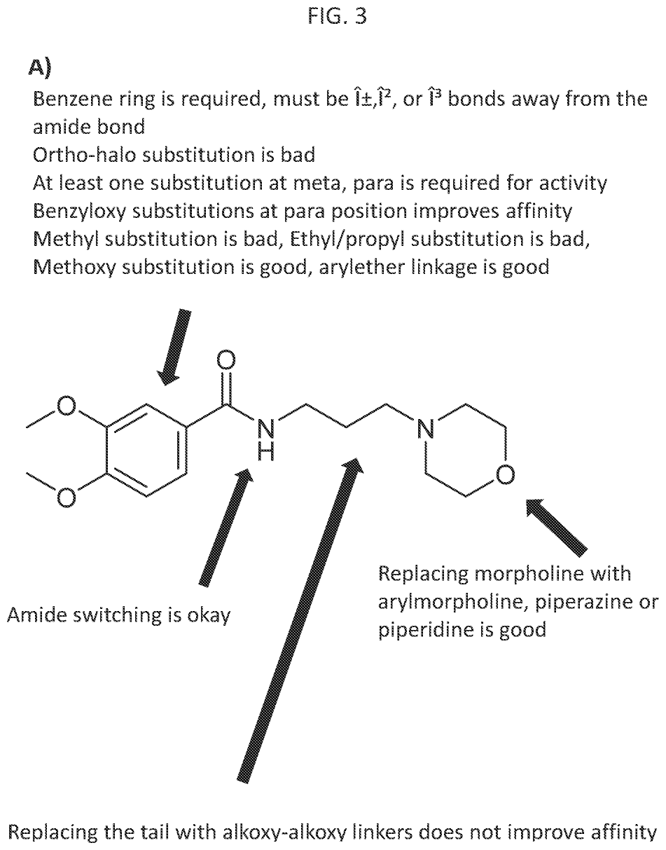

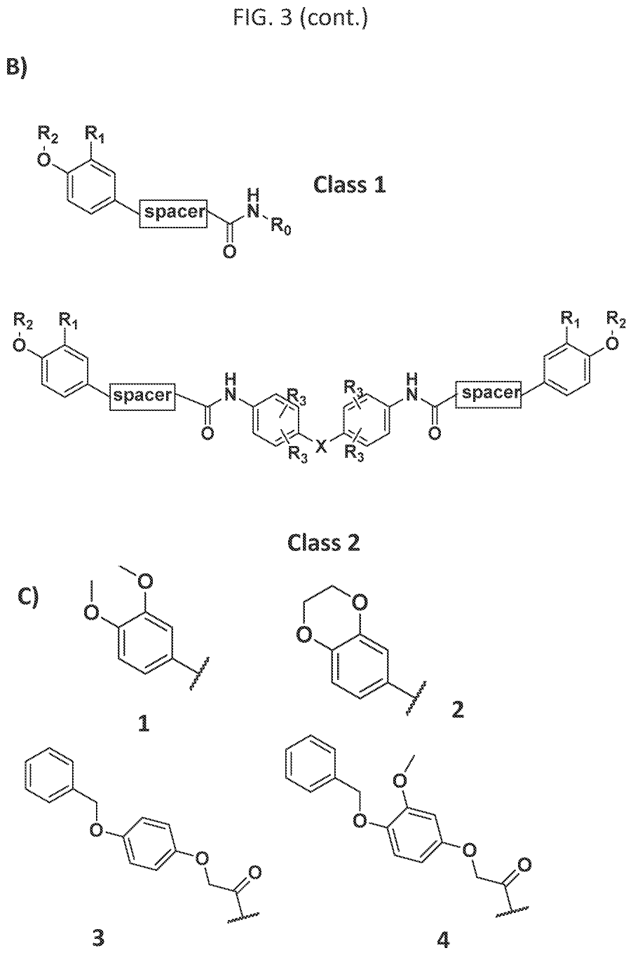

In some aspects, the second grouping (Class II) of compounds are C2-symmetric compounds based on the same scaffolds as Class I analogs (FIG. 3B) and named 2a-2j in FIG. 6. Each class of compounds shares a set of scaffolds like the “head” group of ARD150, shown in FIG. 6. Along with “tail” groups much like those of ARD150, these two groups are permuted to form a broad family of similar compounds while reducing cost and complexity.

In some aspects, the present disclosure concerns a Class I compound reacted with a diamine. In other aspects, the present disclosure concerns one of Formulas Ia, Ib, Ic, or Id linked through an amide to a diamine as set forth in Formula III. In some aspects, the present disclosure concerns one of Formulas Ia, Ib, Ic, or Id linked through an amide to a diamine as set forth in IIIl, IIIb, IIIc, IIId, IIle, or IIIf.

In certain aspects, the present disclosure concerns the ARD150 derivatives of the structures 2a, 2b, 2c, 2d, 2e, 2f, 2g, 2h, 2i, and 2j as set forth in FIG. 6.

In some aspects, the compound has the following structure:

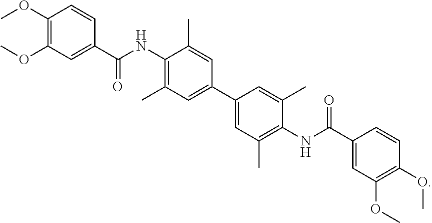

In some aspects, the compound is referred to as 2b as set forth in FIG. 6, or as RWR10 or RWR-10 herein.

It should be understood that the present disclosure not only includes the compounds as described herein, but also salts or deivatives thereof. For example, for solubility and/or administration purposes, it can be desirable to produce a salt form. Such can include ionization of the compound and pairing with a cation/anion to provide the salt. Cations may include aluminum, arginine, lysine, benzathine, magnesium, histidine, lithium, meglumine, potassium, sodium, procaine, triethylaminje, zinc, ethylenediamine, ethanolamine, diethanolamine, choline, chloroprocaine, and calcium. Anions may include, acetate, chloride, aspartate, lactobionate, malate, maleate, mandelate, mesylate, benzenesulfonate, benzoate, besylate, bicarbonate, bitartrate, bromide, methylsulfate, napsylate, nitrate, octanoate, oleate, pamoate, pantothenate, citrate, camsylate, carbonate, decanoate, edetate, esylate, phosphate, polygalacturonate, propionate, fumarate, gluceptate, gluconate, glutamate, glycolate, salicylate, stearate, succinate, sulfate, tartrate, teoclate, tosylate, lactate, isethionate, hexanoate, hydroxynaphthoate, and iodide.

It will also be appreciated that the present disclosure also includes pharmacuetical compositions that include the compounds as set forth herein. Such may include a crystal or amorphous form of the compound or a salt thereof. Such may include additional materials, such as excipients, carriers, surfactants, other active compounds, flavoring agents, vitamins, minerals, and the like. Compositions may comprise other ingredients, known per se by one of ordinary skill in the art, such as pharmaceutically acceptable carriers, excipients, diluents, adjuvants, freeze drying stabilizers, wetting or emulsifying agents, pH buffering agents, gelling or viscosity enhancing additives, and preservatives, depending on the route of administration. Such are described in further detail in Remington: The Science and Practice of Pharmacy, Lippincott Williams & Wilkins, 22nd Ed., 2012; and Ansel's Pharmaceutical Dosage Forms and Drug Delivery Systems, 10^{th} Ed., Philadelphia, PA, 2013.

Examples of pharmaceutically acceptable carriers, excipients or diluents include, but are not limited to demineralised or distilled water; saline solution; vegetable based oils such as peanut oil, arachis oil, safflower oil, olive oil, cottonseed oil, maize oil, sesame oil, or coconut oil; silicone oils, including polysiloxanes, such as methyl polysiloxane, phenyl polysiloxane and methylphenyl polysolpoxane; volatile silicones; mineral oils such as light liquid paraffin oil, or heavy liquid paraffin oil; squalene; cellulose derivatives such as methylcellulose, ethylcellulose, carboxymethylcellulose, carboxymethylcellulose sodium salt, or hydroxypropyl methylcellulose; lower alkanols, for example ethanol or isopropanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrrolidone; agar; carrageenan; gum tragacanth or gum acacia; and petroleum jelly. Typically, the carrier or carriers will form from 10% to 99.9% by weight of the vaccine composition and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

The route of administration can be oral, sublingual, intranasal, transdermal (i.e., applied on or at the skin surface for systemic absorption), ocular, percutaneous, via mucosal administration, or via a parenteral route (intradermal, intramuscular, subcutaneous, intravenous, or intraperitoneal).

The terms “pharmaceutically-acceptable,” “physiologically-tolerable,” and grammatical variations thereof, as they refer to compositions, carriers, excipients, and reagents, are used interchangeably and represent that the materials are capable of administration to or upon a subject without the production of undesirable physiological effects to a degree that would prohibit administration of the composition. For example, “pharmaceutically-acceptable excipient” means, for example, an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. A person of ordinary skill in the art would be able to determine the appropriate timing, sequence and dosages of administration for particular compositions of the present disclosure.

In aspects, preferred examples of such carriers or diluents include, but are not limited to, water, saline, Ringer's solutions, dextrose solution, and 5% human serum albumin. Liposomes and non-aqueous vehicles such as fixed oils can also be used. The use of such media and compounds for pharmaceutically active substances is well known in the art.

Solutions or suspensions used for parenteral, intradermal, or subcutaneous application can include, but are not limited to, the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. Examples of excipients can include starch, glucose, lactose, sucrose, gelatin, malt, rice, flour, chalk, silica gel, water, ethanol, DMSO, glycol, propylene, dried skim milk, and the like. The composition can also contain pH buffering reagents, and wetting or emulsifying agents.

### Methods of Use

As set forth in the working examples herein, it is demonstrated that ARID4B can be responsible for activation of estrogen receptor (ER) signaling, primarily through the ERα. As is also demonstrated herein, targeting the ARID4B gene or expressed gene or the activity thereof can affect ER activity. Accordingly, it is an aspect of the disclosure that methods of reducing or inhibiting ARID4B activity also negatively impact ER activity. Such can be achieved with techniques such as with the administration of siRNA, dsRNA, antibody or actrive fragment(s) thereof or similar.

It is also an aspect of the present disclosure that administration of the compounds as set forth herein impacts ARID4B activity and/or ER activity and/or ERα activity.

In some aspects, the present disclosure concerns administering one or more of the ARD150 derived compounds as set forth herein to a cell. In some aspects, the cell is in vitro. In some aspects, the cell is in vivo. In some aspects, one or more of the ARD150 derived compounds as set forth herein is administered to a subject. As identified herein, ARD150 binds to and inhibits ARDI4B and/or the chromobarrel thereof. Through the identification of derivatives of ARD150 as set forth herein, their application as improved inhibitors and/or more potent inhibitors through their administration to a cell and/or a subject is also contemplated. In some aspects, the present disclosure concerns administration of an effective amount of an ARD150 derivative, wherein an effective amount include an effective dose (ED) or effective concentration (EC) such that the desired response of ARID4B inhibition is achieved. In some aspects, the amount may be of about the ED50 or EC50 or that amount wherein 50% of a studied population demonstrates the desired ARID4B inhibition. In some aspect, the amount administered is above the minimum effective dose, but below the maximum tolerated dose. Such amounts may very between derivatives, but each can be readily determined through straightforward analysis.

In some aspects, the present disclosure concerns administration of at least one of compounds 1a, 1b, 1c, 1d, 1e, 2a, 2b, 2c, 2d, 2e, 2f, 2g, 2h, 2i, 2j, or a combination thereof to a cell, such as a cell within a subject. In some aspects, the compounds can be administered in their native form or as a salt thereof. Administration may include the additional presence of a pharmaceutically effective carrier. Routes of administration in vivo may include oral, sublingual, subdermal, intravenous, intramuscular, inhalation, or other as understood in such endeavors. Examples of acceptable carriers and the like can be found at Remington: The Science and Practice of Pharmacy, 23^{rd} Edition, A. Adejare ed., 2020.

The methods may include administration or providing one or more of the compoisitions as set forth herein to a cell or a subject. In some aspects, the compositions of the present disclosure are provided to a human subject.

In aspects, the compositions of the present disclosure may be administered alone or as a pharmaceutical compoisition. The compositions may be administered by routes such as oral, sublingual, intranasal, transdermal (i.e., applied on or at the skin surface for systemic absorption), ocular, percutaneous, via mucosal administration, or via a parenteral route (intradermal, intramuscular, subcutaneous, intravenous, or intraperitoneal).

As also disclosed herein, it is an aspect that ARID4B is upregulated or overexpressed or overactive in some types of cellular dysplasia, including oncogenesic cells and cancerous cells. Administration of the compounds and compositions described herein can negatively impact hyperplasia, dysplasia, oncogenic growth or tumor growth. The compounds of the present disclosure can be administered alone or in combination with one or more other therapeutic compounds. It will be apparent that as the present disclosure identifies ARID4B aberrant activity in breast tiossue, combining with other active agents for breast cancers can be of a benefit. It should also be apparent that ocmbination with one or more pro-apoptotic agents and/or chemotherapeutic agents and/or antibodies/fragments thereof and/or radiation treamtments. Such can be administered together, independently, over synchronized time courses, and/or over individualized time courses.

A 1^{st} aspect, either alone or in combination with any other aspect herein concerns an ARD150 derivative compound comprising the structure as set forth in Formula (II),

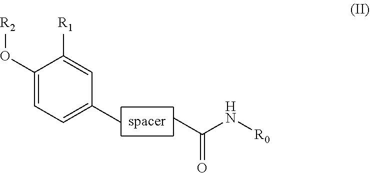

or a salt thereof wherein the spacer is null or —OCH₂; R₁ is null, H, or CH₃; R₂ is H or CH₃; and, R₀ is selected from:

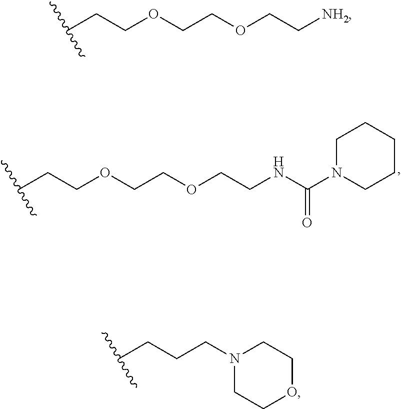 [SMILES: *CCOCCOCCN ; *CCOCCOCCNC(=O)N1CCCCC1 ; "," ; *CCCN1CCOCC1]

or a piperazine with the nitrogen of the depicted amide.

A 2^{nd} aspect, either alone or in combination with any other aspect herein concerns the ARD150 derivative compound of the 1^{st} aspect, wherein the compound is selected from 1a-1e:

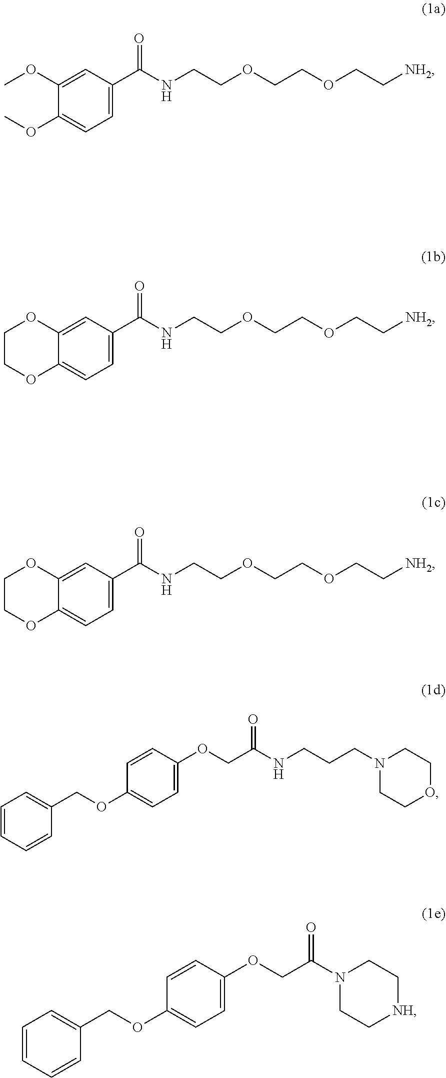 [SMILES: COc1ccc(C(=O)NCCOCCOCCN)cc1OC ; NCCOCCOCCNC(=O)c1ccc2c(c1)OCCO2 ; NCCOCCOCCNC(=O)c1ccc2c(c1)OCCO2 ; O=C(COc1ccc(OCc2ccccc2)cc1)NCCCN1CCOCC1 ; O=C(COc1ccc(OCc2ccccc2)cc1)N1CCNCC1]

or a salt thereof.

A 3^{rd} aspect, either alone or in combination with any other aspect herein concerns the ARD150 derivative compound of the 1^{st} or 2^{nd} aspect, wherein the compound comprises

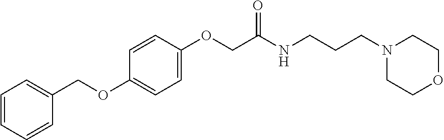 [SMILES: O=C(COc1ccc(OCc2ccccc2)cc1)NCCCN1CCOCC1]

or a salt thereof.

A 4^{th} aspect, either alone or in combination with any other aspect herein concerns the ARD150 derivative compound of the 1^{st} aspect, wherein the composition of Formula II bookends a diamine with a basic structure as set forth in Formula III:

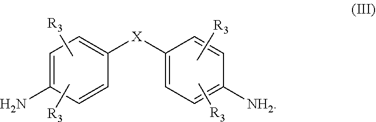

A 5^{th} aspect, either alone or in combination with any other aspect herein concerns the ARD150 derivative compound of the 4^{th} aspect, wherein each R3 is independently null, C₆H₅OC₆H₅CH₂, or CH₃.

A 6^{th} aspect, either alone or in combination with any other aspect herein concerns the ARD150 derivative compound of the 4^{th} aspect, wherein X is null, O, or S—S.

A 7^{th} aspect, either alone or in combination with any other aspect herein concerns the ARD150 derivative compound of the 4^{th} aspect, wherein the diamine is selected from the following structures:

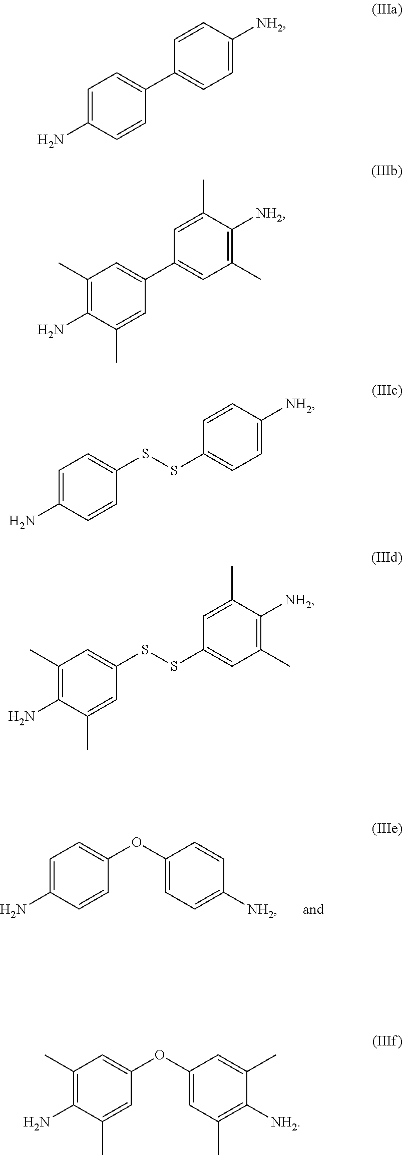 [SMILES: Nc1ccc(-c2ccc(N)cc2)cc1 ; Cc1cc(-c2cc(C)c(N)c(C)c2)cc(C)c1N ; Nc1ccc(SSc2ccc(N)cc2)cc1 ; Cc1cc(SSc2cc(C)c(N)c(C)c2)cc(C)c1N ; Nc1ccc(Oc2ccc(N)cc2)cc1 ; Cc1cc(Oc2cc(C)c(N)c(C)c2)cc(C)c1N]

A 8^{th} aspect, either alone or in combination with any other aspect herein concerns the ARD150 derivative compound of the 7^{th} aspect, wherein the bookended or symmetrical compounds are set forth in Formula IV:

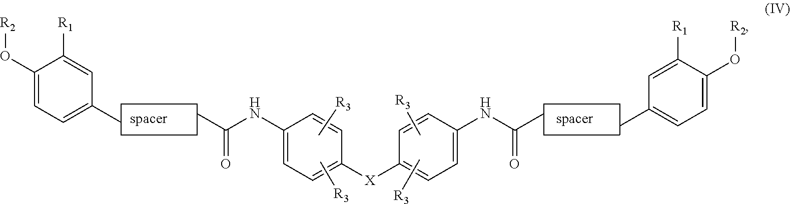

wherein each spacer is independently null or OCH₂; each R₁ is independently null, H, or CH₃; each R₂ is independently H or CH₃; each R3 is independently null, C₆H₅OC₆H₅CH₂, or CH₃; and, X is null, O, or S—S.

A 9^{th} aspect, either alone or in combination with any other aspect herein concerns the ARD150 derivative compound of the 7^{th} aspect, wherein the compounds is selected from compounds 2a-2f as follow:

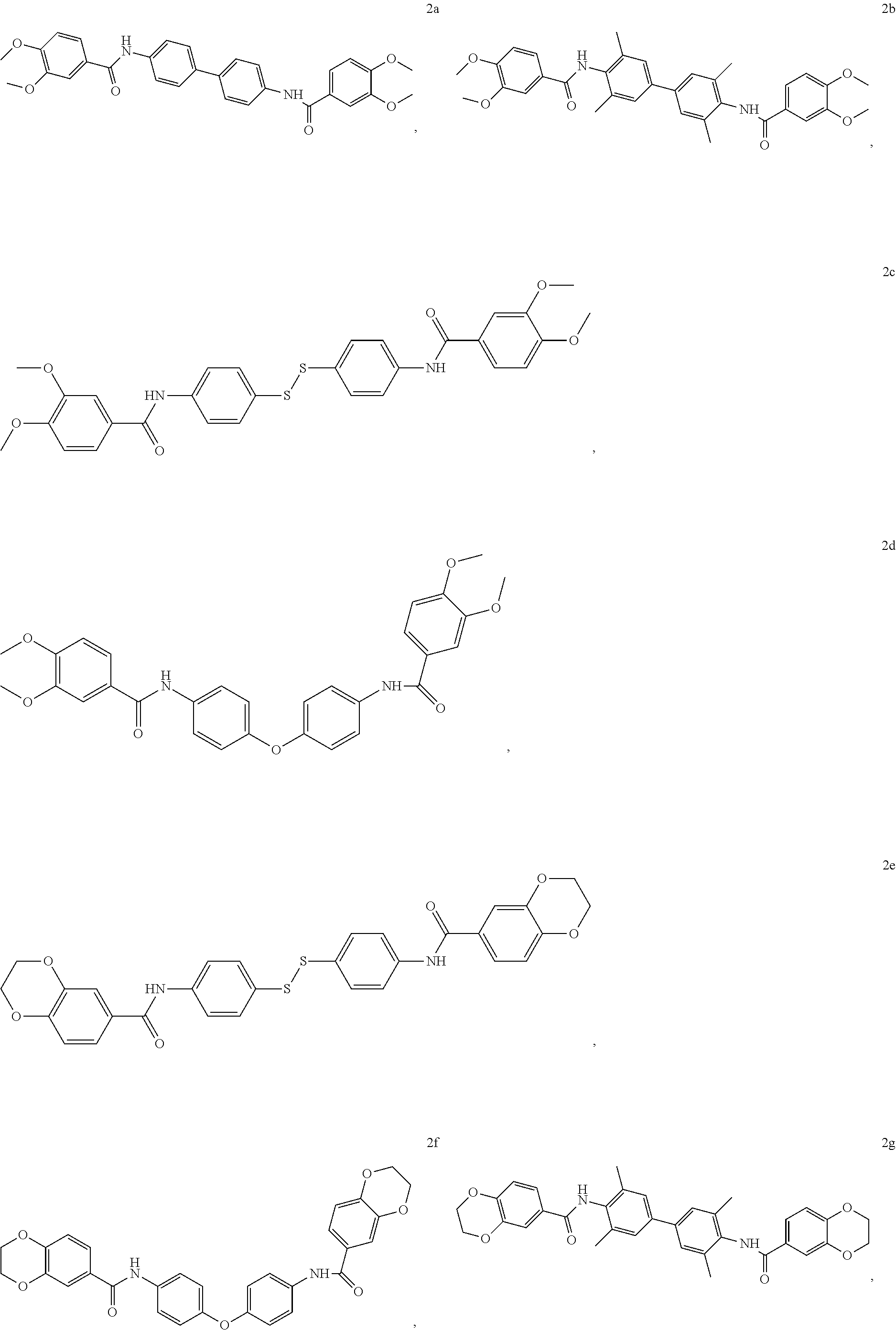

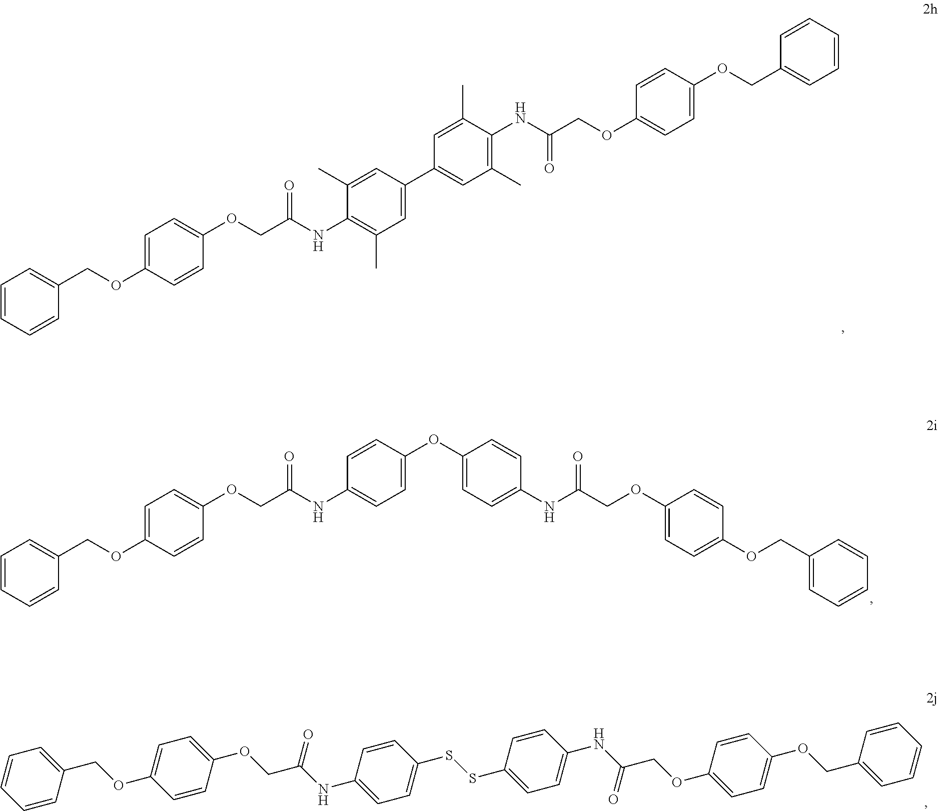 [SMILES: Cc1cc(-c2cc(C)c(NC(=O)COc3ccc(OCc4ccccc4)cc3)c(C)c2)cc(C)c1NC(=O)COc1ccc(OCc2ccccc2)cc1 ; O=C(COc1ccc(OCc2ccccc2)cc1)Nc1ccc(Oc2ccc(NC(=O)COc3ccc(OCc4ccccc4)cc3)cc2)cc1 ; O=C(COc1ccc(OCc2ccccc2)cc1)Nc1ccc(SSc2ccc(NC(=O)COc3ccc(OCc4ccccc4)cc3)cc2)cc1]

or a salt thereof.

A 10^{th} aspect, either alone or in combination with any other aspect herein concerns the ARD150 derivative compound of the 1^{st} or 7^{th} aspect, wherein the compounds comprises

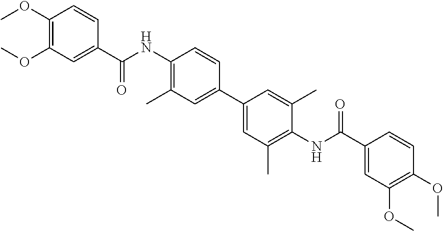

or a salt thereof.

An 11^{th} aspect, either alone or in combination with any other aspect herein concerns the ARD150 derivative compound of the 1^{st} aspect, wherein the compounds comprises

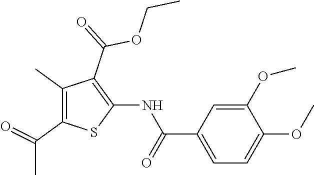 [SMILES: CCOC(=O)c1c(NC(=O)c2ccc(OC)c(OC)c2)sc(C(C)=O)c1C]

or a salt thereof.

A 12^{th} aspect, either alone or in combination with any other aspect herein concerns a pharmecutical composition comprising the compouind of any of apects 1 to 11 and a pharmaceutically acceptable carrier.

A 13^{th} aspect, either alone or in combination with any other aspect herein concerns the pharmaceutical composition of the 12^{th} aspect, further compriusing an excipient.

A 14^{th} aspect, either alone or in combination with any other aspect herein concerns a method for targeting AT-rich interactive domain 4B (ARID4B) in a cell, comprising administering the ARD150 derivative compound of any one of aspects 1-13 to a cell.

A 15^{th} aspect, either alone or in combination with any other aspect herein concerns the method of the 14^{th} aspect, wherein the cell is a cancer cell.

A 16^{th} aspect, either alone or in combination with any other aspect herein concerns the method of the 14^{th} aspect, wherein the cell is in vivo.

A 17^{th} aspect, either alone or in combination with any other aspect herein concerns a method for treating cellular dysplasia in breast tissue of a subject comprising administering the compound of any of aspects 1 to 12 to the subject.

A 18^{th} aspect, either alone or in combination with any other aspect herein concerns the method of the 17^{th} aspect, wherein the compound is administered by a route selected from parenteral, topical, intravenous, oral, subcutaneous, sublingual, intraarterial, intradermal, transdermal, rectal, intracranial, intrathecal, intraperitoneal, intranasal; vaginally; intramuscular route or as inhalants.

### EXAMPLES

ARID4B is amplified and expression is elevated in breast cancer.

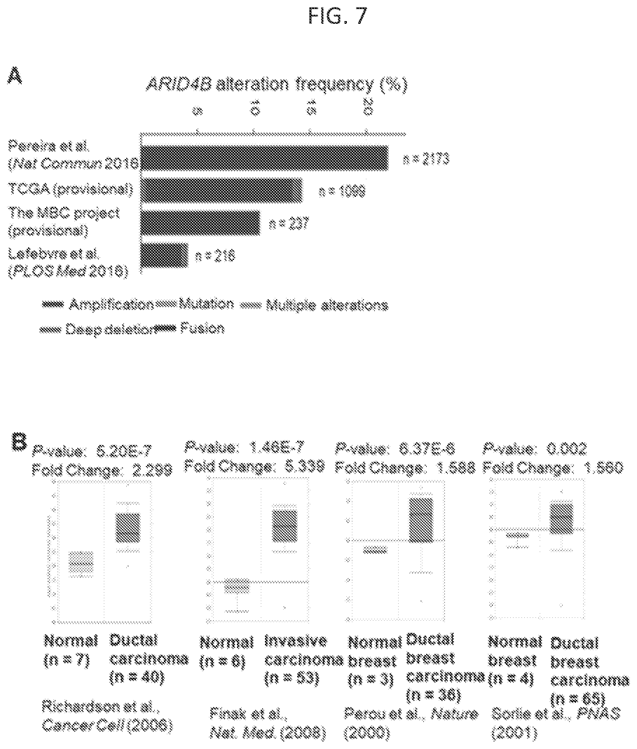

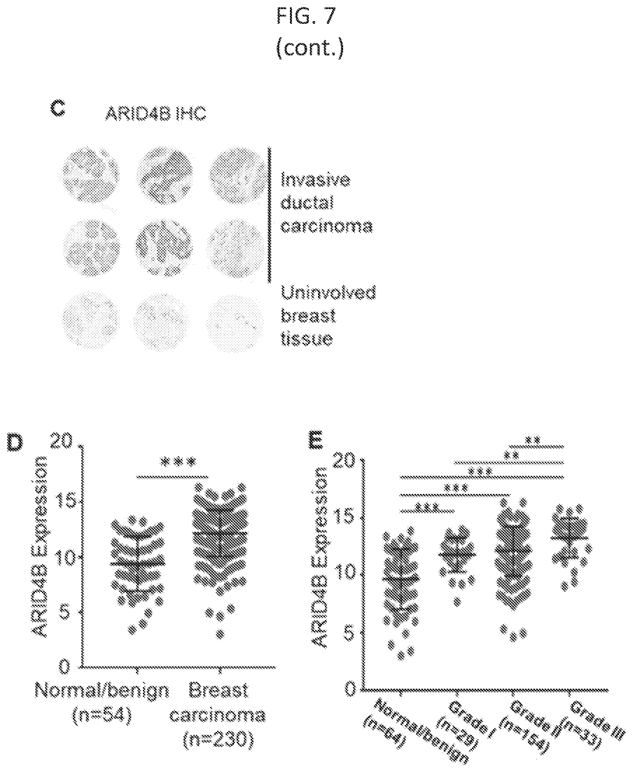

Analysis of breast cancer genomes in The Cancer Genome Atlas (TCGA) and other breast cancer datasets showed that ARID4B is amplified in breast cancer (up to 22%) (FIG. 7A). In addition, transcriptome analysis revealed expression of ARID4B is elevated in breast cancers compared to controls (FIG. 7B). Immunohistochemical (IHC) staining using breast tumor arrays confirmed elevated ARID4B protein levels in tumors compared to normal/benign breast tissues (FIGS. 7C & 7D), and higher levels of ARID4B in Grade III tumor compared with Grades I and II (FIG. 7E).

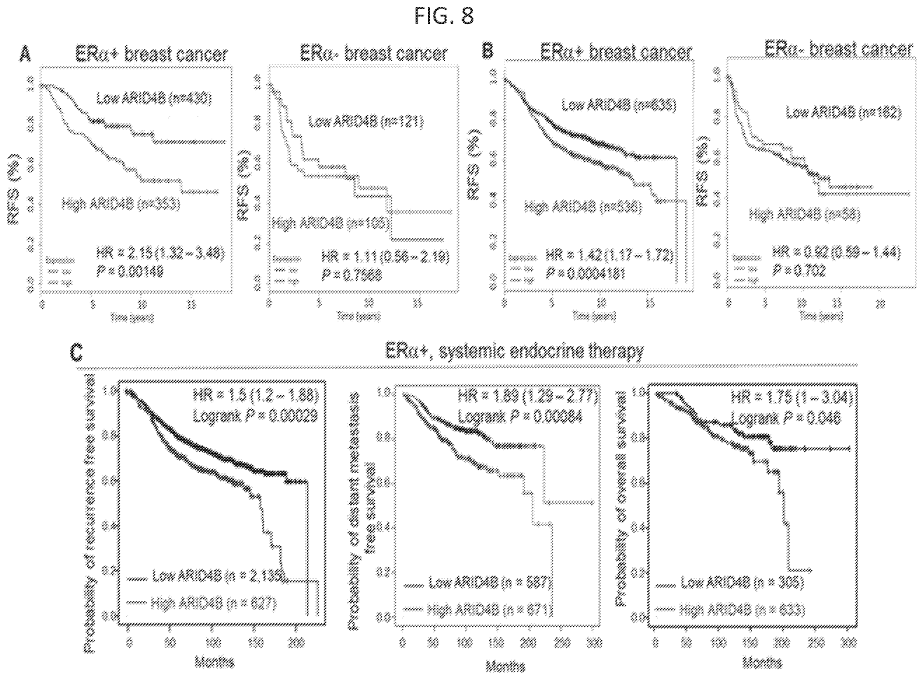

Kaplan-Meier survival analysis revealed that elevated ARID4B expression is associated with decreased recurrence-free survival (RFS), particularly evident in patients with ERα+ and not ERα− breast cancers (FIGS. 8A & 8B). Further, elevated ARID4B expression is associated with unfavorable clinical outcomes in patients with ERα+ breast cancer who received systemic endocrine therapy (FIG. 8C).

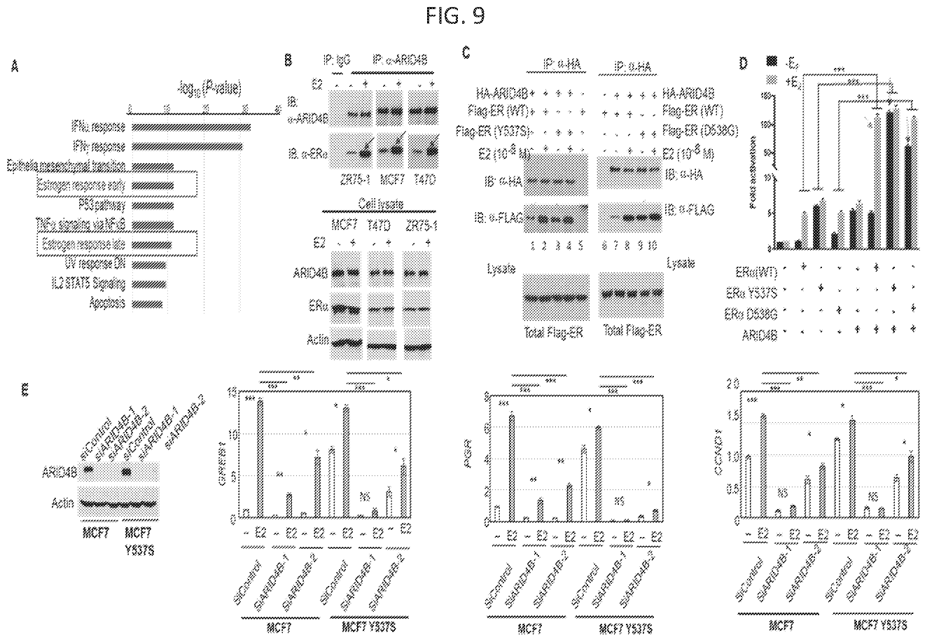

To determine whether ARID4B is involved in ERα signaling, RNA-seq and gene set enrichment analysis (GSEA) were performed. The results showed the ER□ signaling pathway to be the top pathway affected by knockdown of ARID4B in MCF7 cells (FIG. 9A). To directly test whether ARID4B functions as a transcription co-activator for ERα, it was investigated whether ARID4B interacts with ERα by co-immunoprecipitation (Co-IP) assays. A weak ligand-independent interaction between endogenous ARID4B with ERα was found and this interaction was enhanced by E2 in ERα-positive MCF7, T47D, and ZR75-1 cells (FIG. 9B, arrows). While rare in primary breast cancer, mutations of the ERα gene (ESR1) are frequent in ERα+ metastatic cancer. Increasing evidence suggests that these mutations confer endocrine resistance in patients with advanced disease. Since ARID4B interacts with wild-type ERα, whether ARID4B also interacts with the most constitutively active ERα Y537S and D538G mutants by Co-IP was tested. Consistent with results in FIG. 9B, a ligand-independent interaction between ARID4B and wild-type ERα was detected, which was further stimulated by E2 (FIG. 9C, lanes 1 and 2, 7 and 8). Interestingly, a ligand-independent interaction between ARID4B, the Y537S and D538G mutants was detected (FIG. 9C, lane 1 vs. 3, and 7 vs. 9), which was further increased by E2 (lanes 4 and 10). To test whether the enhanced interaction effects ligand-independent activation of Y537S and D538G mutant receptors, ERE-driven reporter gene assays were performed. ARID4B co-activated with ERα on a reporter gene containing the ER-responsive element (ERE-Luc) (FIG. 9D, arrow). Importantly, ARID4B stimulated the ligand-independent activity of Y537S and D538G mutants to almost the same extent as in the presence of E2 (FIG. 9D, arrows). CRISPR-Cas9-mediated introduction of a mutation in the native ESR1 gene locus in MCF7 cells generated an isogenic mutant MCF7 Y537S. These MCF7 Y537S mutant cells express high levels of select ERα target genes, including PGR, GREB1, and CCND1, exhibit E2-independent growth, and are resistant to tamoxifen and fulvestrant (kindly provided by Dr. S. Ali, Imperial College of London). Consistent with RNA-seq and GSEA analyses (FIG. 9A, and not shown), ARID4B knockdown inhibited E2 induction of PGR, CCND1 and GREB1 in MCF7, T47D and ZR75-1 cells (FIG. 9E and not shown). Importantly, ARID4B knockdown strongly inhibited ligand-independent, constitutive expression of GREB1, PGR, and CCND1 in MCF7 Y537S cells (FIG. 9E).

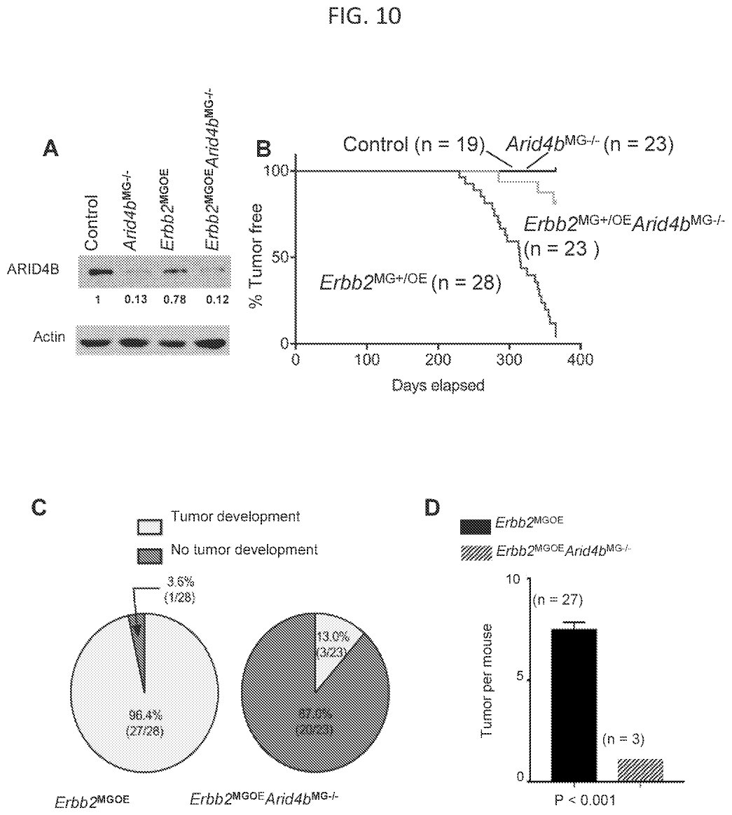

It was next determined whether there exists a causal role of ARID4B in mammary gland tumorigenesis in vivo, using MMTV-Cre; MMTV-Erbb2 mice (JAX) that express Erbb2 (Erbb2^{MGOE}) and develop mammary gland tumors. Erbb2^{MGOE} mice were crossed with Arid4b^{flox/flox} mice to generate mice expressing Erbb2 but carrying a mammary gland-specific Arid4b deletion (Erbb2^{MGOE}Arid4b^{MG-/-}) (FIG. 10A). 27 out of 28 Erbb2^{MGOE} mice developed spontaneous tumors with 50% incidence at 42 wks. In contrast, only 3 out of 23 (13%) Erbb2^{MGOE}Arid4b^{MG-/-} mice developed tumors at 60 wks (FIGS. 10B, 10C). Moreover, each Erbb2^{MGOE} mouse developed multiple tumors, but only single tumor in each of the three tumor-bearing Erbb2^{MGOE}Arid4b^{MG-/-} mice (FIG. 4D). Knockout of Arid4b (Arid4b^{MG-/-}) did not affect mammary gland development (not shown), and control and Arid4b^{MG-/-} mice did not develop tumors (FIG. 10B).

These results identified ARID4B as a novel coactivator for ERα and show that mammary gland-specific ablation of Arid4b in mice inhibits tumorigenesis. Interestingly, ARID4B not only activated the wild-type ERα, but also interacted with constitutively active ERα mutants in a ligand-independent manner and stimulated their activity to the full extent as in the presence of E2. Collectively, these results strongly suggest that activation of ERα by ARID4B may drive breast cancer development and progression to antiestrogen resistant cancer.

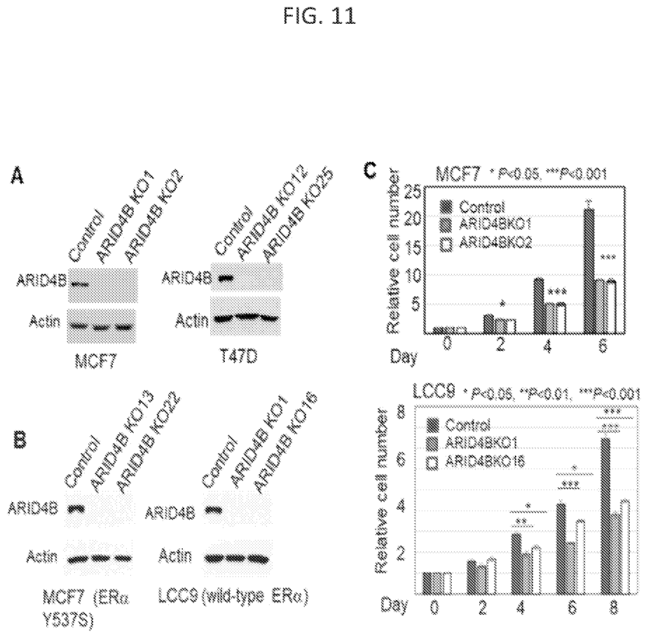

ARID4B knockout (ARID4B KO) cells were generated in ERα+ breast cancer cells, including the E2-dependent MCF7 and T47D (FIG. 11A), and E2-independent MCF7 Y537S and LCC9 cells (anti-estrogen acquired resistant MCF7 variant, which express wild-type ERα, provided by Dr. R. Clarke) (FIG. 11B). Knockout of ARID4B reduced the proliferation of these breast cancer cells (FIG. 11C and not shown), supporting an important role of ARID4B in breast cancer.

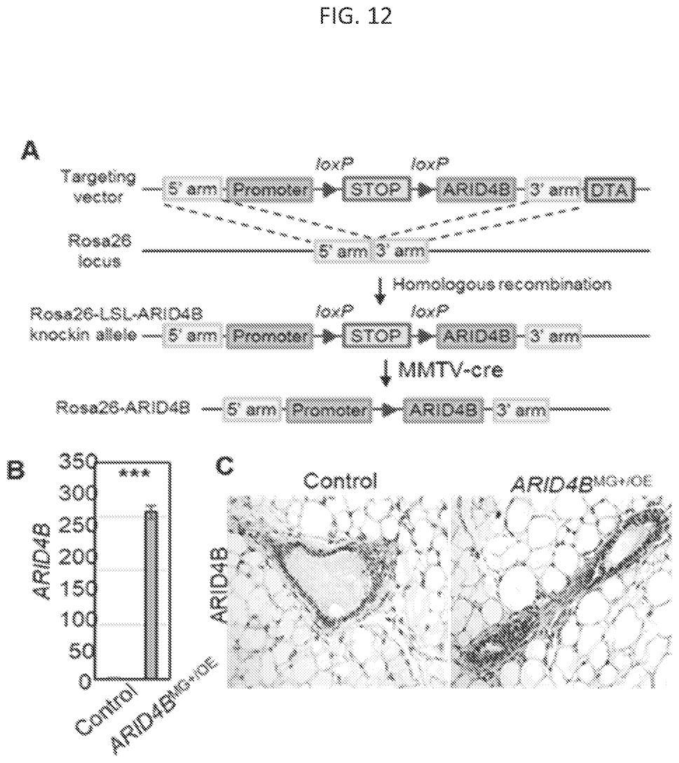

ARID4B knock-in mice that overexpress ARID4B in a mammary gland-specific manner (ARID4B^{MGOE}) (FIG. 12) were generated to investigate the following ARID4B function. To generate mice that over-express human ARID4B (˜90% aa identity with mouse's) in a mammary gland-specific manner (ARID4B^{MG+/OE}), we knocked in the targeting vector carrying the ARID4B cDNA to the Rosa26 locus (Rosa26-LSL-ARID4B) (FIG. 12A). These mice were crossed with MMTV-Cre to remove the stop codon and to generate the ARID4B^{MG+/OE} mice that overexpress ARID4B, a powerful tool complementary to the KO mouse model in our proposed study. qRT-PCR using mammary gland primary cells and IHC confirmed over-expression of ARID4B (FIGS. 12B, 12C).

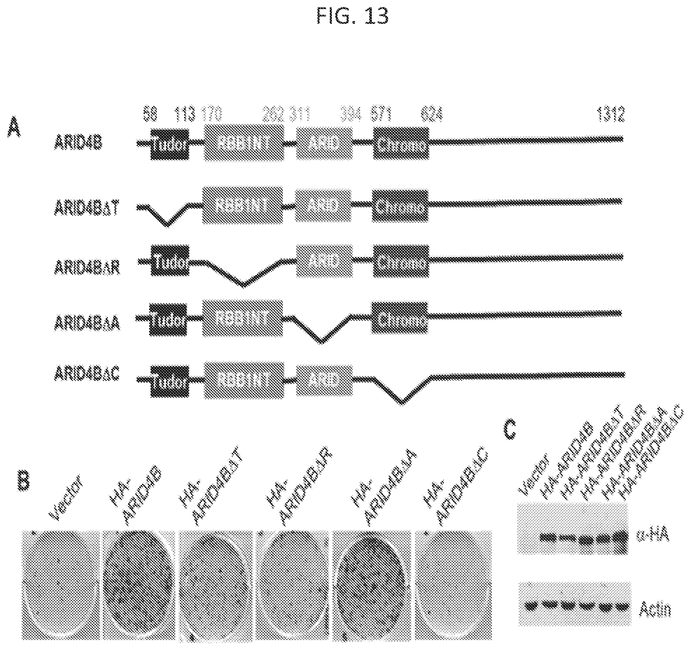

ARID4B contains a Tudor domain, a RBBP1 N-terminal domain (RBB1NT, also known as PWWP domain for the conserved Pro-Trp-Trp-Pro motif), an ARID domain (a putative DNA binding domain), and a chromo domain at its N-terminus. Tudor and chromo domain bind methylated histone. The function of RBB1NT domain remains unclear, but it was proposed to mediate protein-protein interaction and binding of methylated histone. To determine which domain(s) is important for the biological function of ARID4B, mutants devoid of the Tudor domain (ΔT, aa58-113), RBB1NT (ΔR, aa170-262,), ARID domain (ΔA, aa311-394), and chromo domain (ΔC, aa571-624) were created by site-directed mutagenesis (confirmed by Sanger sequencing, FIG. 13A). Colony formation assays were performed to assess the ability of these mutants to promote proliferation in MCF7 ARID4B KO cells. MCF7 ARID4B KO cells (FIG. 11) were transfected with vector, full-length ARID4B or ARID4B deletion mutants and selected with hygromycin (400 μg/ml) for 2 weeks. Colonies were visualized by staining with crystal violet. Compared to vector control, expression of full-length ARID4B promoted cell proliferation/survival in MCF7 ARID4B KO cells (FIG. 13B). Similar to full-length ARID4B, the ARID4B mutant with the ARID domain deletion, but not those with deletion of Tudor, RBB1NT, or chromo domains, promoted cell proliferation/survival (FIG. 13B). A parallel experiment confirmed comparable expression between wild-type and ARID4B mutants (FIG. 13C).

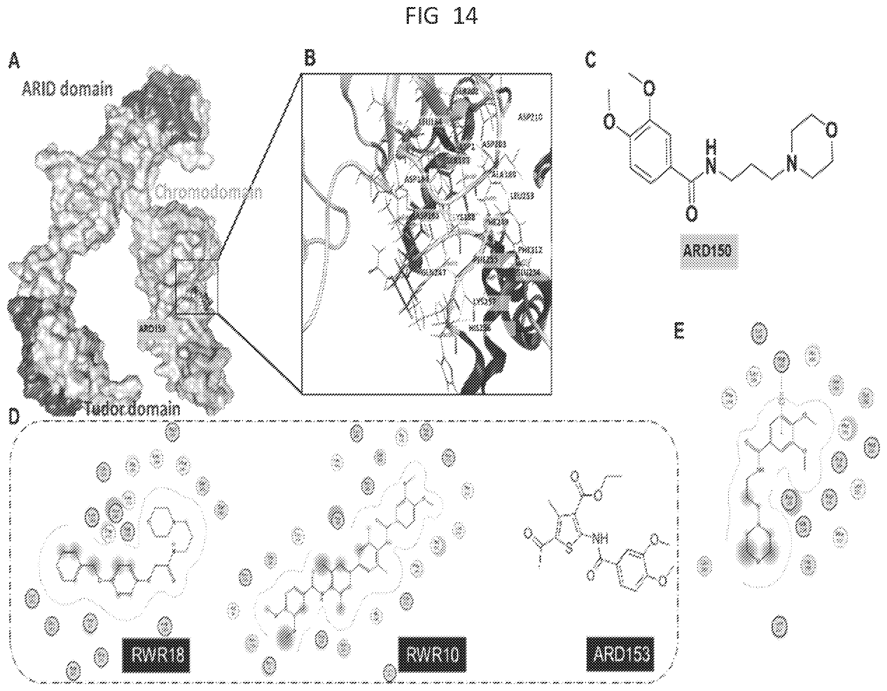

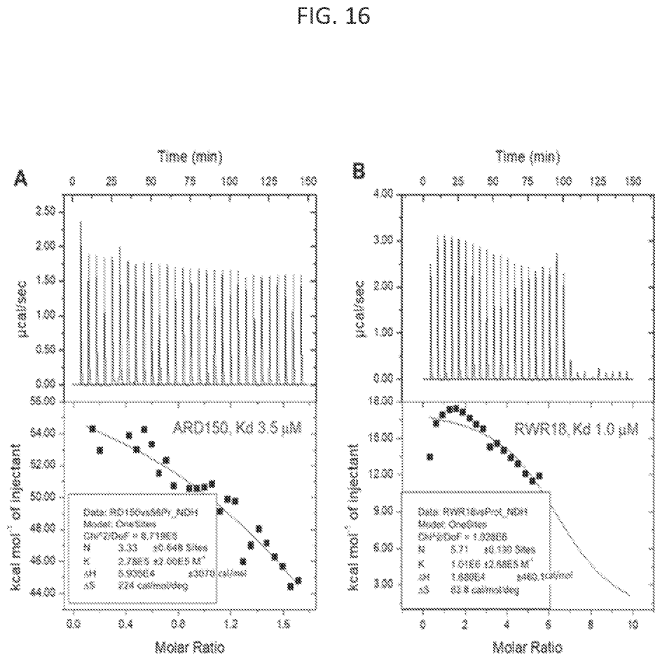

It was then undertaken to identify compounds that inhibit ARID4B, focusing on the chromodomain therein. First, the structure of ARID4B was created using a homology model based on an optimized computational platform that combines the iterative threading assembly refinement (TASSER) and SWISSMODEL (FIG. 14A-B). Protein structure prioritization was based on protein-model accuracy, quantitatively measured C-score, TM-score and RMSD value. Next, in silico screening of 160,000 drug-like diverse small molecules from MOE's chemical repository combined with biophysical characterization including isothermal calorimetry (ITC, FIG. 16) and circular dichroism (data not shown) of identified compound ARD150 with recombinant ARID4B chromodomain informed chemical synthesis of diverse scaffolds.

### Compound Development

These data summarize different approaches to synthesize and characterize the two classes (Class I and II) of novel, potent analogs of ARD150. It will be appreciated that through the use of the identified sets of “head” and “tail” groups, a wide variety of analogs can be created.

### General Procedure for the Synthesis of Class I Analogs

### Synthesis of N-Boc Intermediates, Compounds 1c and 1d

To a doubly purged Schlenk flask equipped with a stir bar was added the acid (1 equiv.), diamine (1 equiv.), EDC (2.5 equiv.), HOBt (2.5 equiv.), DIPEA (2.5 equiv.), and DMF (5 mL). This process formed an off-white heterogeneous mixture, which was then allowed to continually stir at room temperature under inert conditions for 24h.

The mixture was diluted with water, centrifuged, and extracted with DCM (5 mL×2), then the combined organic layers were dried over anhydrous magnesium sulfate and filtered. The resultant filtrate was removed in vacuo to afford the crude amide, which was then purified via chromatography (silica; 1-5% methanol in DCM (mobile phase)) to yield the N-Boc intermediate compound.

### Synthesis of Compounds 1a, 1b, and 1e

To a solution of the starting material (1 equiv.) in dichloromethane (5 mL) was added trifluoroacetic acid (5 mL, approx. 10 eq), and the mixture was stirred at room temperature for one hour. After this time, the reaction was concentrated under reduced pressure to remove most of the excess TFA.

The resulting viscous oil was dissolved in dichloromethane (12 mL), washed several times with diethyl ether (ca. 30 mL), and concentrated to yield an off-white precipitate. To this precipitate was added 20 mL of saturated sodium bicarbonate solution, then extracted with DCM, dried with magnesium sulfate, filtered, and concentrated under reduced pressure to yield the target compound as an off-white solid.

### General Procedure for the Synthesis of Class II Analogs

### Synthesis of Compounds 2a, 2c, 2e

A doubly purged Schlenk flask equipped with a stir bar was added the acid (2 equiv.), diamine (1 equiv.), EDC (2.5 equiv.), HOBt (2.5 equiv.), DIPEA (2.5 equiv.), and DMF (5 mL). This process formed an off-white heterogeneous mixture, which was then allowed to continually stir at room temperature under inert conditions for 24h.

The mixture was diluted with water, centrifuged, and extracted with DCM (5 mL×2), then the combined organic layers were dried over anhydrous magnesium sulfate and filtered. The resultant filtrate was removed in vacuo to afford the crude amide, which was then purified via chromatography (silica; 1-5% methanol in DCM (mobile phase)) to yield the target compound.

### Synthesis of Compounds 2b, 2d, 2f-2j

To a round bottom flask equipped with a stir bar was added the acid (2 equiv.), aryl diamine (1 equiv.), HATU (2.5 equiv.), and DMF (5 mL) This formed an off-white heterogeneous mixture, which was then allowed to stir at room temperature for 24h continually.

The mixture was diluted with water and extracted with DCM (5 mL×2), and the combined organic layers were dried over anhydrous magnesium sulfate and filtered. The resultant filtrate was removed in vacuo to afford the crude amide, which was then purified via chromatography (silica; 30% ethyl acetate (mobile phase)) to yield the target compound.

### Characterization Techniques

Reactions were monitored throughout via (silica) TLC with a workable solvent system as the mobile phase (often either 30-50% ethyl acetate in hexane or 1-5% methanol in DCM seemed to work). Likewise, compounds were purified using corresponding column chromatographic methods using the same solvent system for its TLC. A given compound was deemed synthesized if ¹H and ¹³C NMR was on point. Each compound was deemed pure and ready for biological studies if its purity (determined via HPLC) was greater than or equal to 97%.

### Results

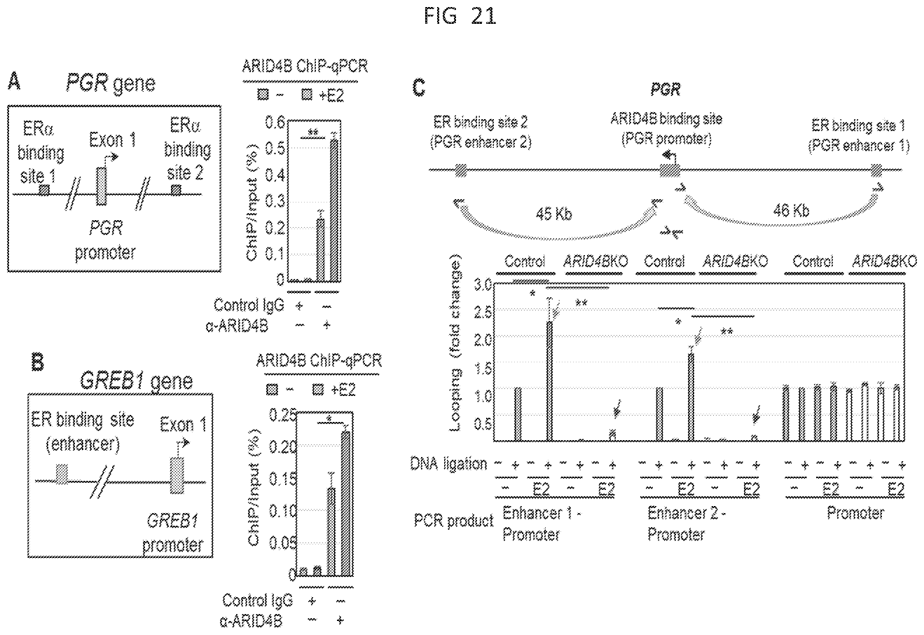

As identified herein, an iterative scoring process was used to develop new variants of ARD150 to minimize the computational resources required. Analogs of ARD150 were generated by optimizing the critical interactions within the chromobarrel domain pocket, which were then re-docked and scored. The highest 1% of these compounds were further studied to become scaffolds. Molecular modeling of a library of compounds against the ARID4B protein revealed three novel classes of compounds better at targeting the protein than ARD150. In addition, docking results and structure-docking score relationships have shown that certain modifications could improve affinity for the ARID4B protein, as shown in FIG. 3A. Fifteen novel analogs of ARD150 have been successfully synthesized, as shown above in FIG. 6. Accompanying ¹H/¹³C NMR spectra and HPLC chromatograms are supplied in below and in FIGS. 7-49. Analysis of NMR spectra is as follows:

N-Boc Intermediate for Compound 1a:

¹H NMR (400 MHZ, Chloroform-d) δ 7.46 (s, 1H), 7.33 (d, J=7.9 Hz, 1H), 6.85 (d, J=8.4 Hz, 1H), 3.91 (d, J=5.9 Hz, 6H), 3.66 (s, 8H), 3.54 (t, J=5.3 Hz, 2H), 3.29 (t, J=5.2 Hz, 2H), 1.42 (s, 9H).

¹³C NMR (101 MHz, CDCl₃) δ 167.21, 156.07, 151.74, 148.94, 127.06, 119.55, 110.78, 110.25, 79.55, 70.21, 56.01, 39.90, 28.40.

Compound 1a:

¹H NMR (400 MHZ, Chloroform-d) 7.44-7.31 (m, 3H), 6.77 (d, J=8.2 Hz, 1H), 6.12 (s, 1H), 3.81 (s, 6H), 3.66-3.48 (m, 10H), 3.03 (dd, J=7.5, 4.3 Hz, 2H).

¹³C NMR (101 MHz, CDCl₃) δ 168.28, 152.01, 148.87, 126.20, 120.25, 110.45, 70.08, 70.03, 69.93, 66.55, 55.99, 54.10, 42.29, 39.90, 39.62

N-Boc Intermediate of Compound 1b:

¹H NMR (400 MHZ, Chloroform-d) δ 7.35 (d, J=11.6 Hz, 2H), 6.90 (d, J=8.4 Hz, 1H), 6.60 (s, 1H), 5.04 (s, 1H), 4.30 (d, J=2.3 Hz, 4H), 3.66 (d, J=6.7 Hz, 8H), 3.58 (t, J=5.2 Hz, 2H), 3.32 (s, 2H), 1.46 (s, 9H).

¹³C NMR (101 MHz, CDCl₃) δ 166.75, 156.01, 146.40, 143.29, 127.89, 120.51, 117.21, 116.47, 79.41, 70.28, 64.54, 64.21, 40.40, 39.65, 28.41.

Compound 1b:

¹H NMR (400 MHZ, DMSO-d6) δ 8.36 (s, 1H), 7.87 (s, 3H), 7.37 (d, J=14.2 Hz, 2H), 6.91 (d, J=8.3 Hz, 1H), 4.28 (s, 4H), 3.62-3.49 (m, 8H), 3.39 (q, J=5.9 Hz, 2H), 2.96 (d, J=5.3 Hz, 2H).

¹³C NMR (101 MHz, DMSO) δ 159.03, 158.68, 146.42, 143.35, 127.87, 121.06, 117.17, 116.68, 70.15, 69.37, 67.14, 64.78, 64.45.

Compound 1c:

¹H NMR (400 MHZ, Chloroform-d) δ 7.34 (s, 1H), 7.31 (s, 1H), 6.88 (d, J=8.4 Hz, 1H), 6.64 (s, 1H), 4.29-4.25 (m, 4H), 3.71-3.54 (m, 12H), 3.41 (t, J=5.1 Hz, 2H), 3.34-3.25 (m, 4H), 3.15 (s, 1H), 1.54 (d, J=9.2 Hz, 9H).

¹³C NMR (101 MHz, CDCl₃) δ 166.74, 157.52, 146.44, 143.33, 127.82, 120.48, 117.23, 116.48, 70.52, 70.23, 69.82, 64.54, 64.22, 47.96, 45.15, 40.65, 39.68, 25.78, 25.45, 24.77, 24.27.

Compound 1d:

¹H NMR (400 MHZ, Chloroform-d) δ 7.37-7.22 (m, 5H), 6.81 (s, 4H), 4.94 (s, 2H), 4.57 (s, 2H), 3.57-3.45 (m, 4H), 3.34 (d, J=11.3 Hz, 4H), 1.39 (s, 9H).

¹³C NMR (101 MHZ, CDCl₃) δ 166.96, 154.51, 153.73, 152.00, 137.10, 128.58, 127.96, 127.49, 115.97, 115.53, 80.39, 76.73, 70.64, 68.54, 45.34, 42.00, 28.38.

Compound 1e:

¹H NMR (400 MHZ, DMSO-d6) δ 8.91 (s, 1H), 7.49-7.31 (m, 5H), 6.98-6.84 (m, 4H), 5.04 (s, 2H), 4.79 (s, 2H), 3.66 (s, 5H), 3.13 (d, J=25.9 Hz, 4H).

¹³C NMR (101 MHz, DMSO) δ 166.87, 158.92, 158.57, 153.17, 152.54, 137.79, 128.87, 128.21, 128.06, 116.02, 70.09, 66.77.

Compound 2a:

¹H NMR (400 MHZ, Chloroform-d) δ 8.11 (d, J=8.4 Hz, 2H), 7.98 (dd, J=8.5, 2.1 Hz, 2H), 7.69 (d, J=2.0 Hz, 2H), 7.58-7.53 (m, 2H), 7.50-7.43 (m, 4H), 7.03 (d, J=8.5 Hz, 2H), 4.02 (s, 6H), 3.98 (s, 6H).

¹³C NMR (101 MHz, CDCl₃) δ 162.46, 155.22, 149.32, 143.61, 128.93, 128.71, 125.64, 124.83, 120.58, 116.70, 112.55, 110.88, 108.47, 56.24.

Compound 2b:

¹H NMR (400 MHZ, DMSO-d6) δ 9.67 (s, 2H), 7.69 (d, J=8.4 Hz, 2H), 7.61 (s, 2H), 7.47 (s, 4H), 7.30 (s, 1H), 7.10 (d, J=8.6 Hz, 2H), 3.85 (s, 12H), 2.26 (s, 12H).

¹³C NMR (101 MHz, DMSO) δ 164.97, 152.03, 138.40, 136.57, 135.39, 126.96, 126.34, 121.25, 111.49, 56.07, 18.75.

Compound 2c:

¹H NMR (400 MHZ, Chloroform-d) δ 8.10 (d, J=8.4 Hz, 2H), 7.98 (dd, J=8.5, 2.1 Hz, 2H), 7.69 (d, J=2.0 Hz, 2H), 7.58-7.53 (m, 2H), 7.50-7.42 (m, 4H), 7.03 (d, J=8.5 Hz, 2H), 4.02 (s, 6H), 3.98 (s, 6H).

¹³C NMR (101 MHz, CDCl₃) δ 162.47, 155.22, 149.32, 143.59, 128.71, 125.64, 124.84, 120.57, 116.70, 112.55, 110.88, 108.47, 56.32.

Compound 2d:

¹H NMR (400 MHZ, DMSO-d6) δ 10.09 (s, 2H), 7.75 (d, J=9.1 Hz, 4H), 7.64-7.58 (m, 2H), 7.53 (d, J=2.0 Hz, 2H), 7.08 (d, J=8.6 Hz, 2H), 7.01 (d, J=9.0 Hz, 4H), 3.84 (s, 12H).

¹³C NMR (101 MHZ, DMSO) δ 165.23, 153.25, 152.07, 148.77, 135.22, 127.39, 122.67, 121.44, 119.04, 111.45, 56.10.

Compound 2e:

¹H NMR (400 MHZ, DMSO-d6) δ 8.77 (s, 1H), 8.55 (s, 1H), 7.52 (dd, J=8.4, 4.4 Hz, 4H), 7.45 (d, J=2.0 Hz, 2H), 7.43 (d, J=2.1 Hz, 2H), 7.38 (d, J=2.0 Hz, 3H), 6.94 (d, J=8.4 Hz, 3H), 4.31 (dd, J=4.4, 3.0 Hz, 8H).

¹³C NMR (101 MHz, DMSO) δ 167.19, 151.60, 147.92, 143.47, 140.08, 135.11, 129.34, 124.21, 123.41, 121.20, 118.60, 117.52, 64.89, 64.36.

Compound 2f:

¹H NMR (400 MHZ, DMSO-d6) δ 8.78 (s, 1H), 8.55 (s, 1H), 7.52 (dd, J=8.4, 4.4 Hz, 4H), 7.44 (dd, J=8.4, 2.1 Hz, 5H), 7.38 (d, J=2.0 Hz, 4H), 6.94 (d, J=8.4 Hz, 5H), 4.33-4.30 (m, 8H).

¹³C NMR (101 MHz, DMSO) δ 167.19, 151.61, 147.92, 143.47, 140.09, 135.11, 129.34, 124.21, 123.41, 121.20, 118.60, 117.52, 64.89, 64.36.

Compound 2g:

¹H NMR (400 MHZ, DMSO-d6) δ 9.58 (d, J=33.7 Hz, 2H), 7.59-7.54 (m, 3H), 7.44 (s, 3H), 7.29 (s, 1H), 7.17 (s, 1H), 7.00 (d, J=8.3 Hz, 2H), 4.32 (s, 8H), 2.24 (s, 12H).

¹³C NMR (101 MHZ, DMSO) δ 167.20, 164.63, 146.77, 143.49, 138.46, 136.55, 136.18, 135.33, 127.71, 126.33, 125.17, 123.41, 121.56, 121.40, 118.60, 117.52, 117.37, 117.05, 64.88, 64.51, 18.70.

Compound 2h:

¹H NMR (400 MHZ, DMSO-d6) δ 9.49 (s, 2H), 7.45-7.34 (m, 14H), 7.00 (s, 8H), 5.07 (s, 4H), 4.67 (s, 4H), 2.16 (s, 12H).

¹³C NMR (101 MHz, DMSO) δ 167.31, 153.35, 152.46, 138.49, 137.78, 136.28, 128.86, 128.10, 126.31, 116.15, 70.10, 18.68.

Compound 2i:

¹H NMR (400 MHZ, Chloroform-d) δ 7.37-7.22 (m, 14H), 6.95-6.77 (m, 12H), 4.94 (d, J=2.4 Hz, 4H), 4.49 (d, J=3.4 Hz, 4H).

¹³C NMR (101 MHz, CDCl₃) δ 166.48, 153.63, 152.32, 151.75, 137.19, 128.62, 128.59, 127.97, 127.93, 127.48, 121.91, 119.30, 116.13, 115.95, 115.87, 115.82, 115.71, 70.64, 68.31, 36.66, 35.76.

Compound 2j:

¹H NMR (400 MHZ, DMSO-d6) δ 10.19 (s, 2H), 7.68 (d, J=8.8 Hz, 4H), 7.52-7.30 (m, 15H), 7.00-6.91 (m, 8H), 5.04 (s, 4H), 4.63 (s, 4H).

¹³C NMR (101 MHZ, DMSO) δ 167.50, 153.37, 152.41, 138.99, 137.75, 130.61, 130.32, 128.86, 128.10, 120.95, 116.14, 70.09, 68.32.

### DISCUSSION

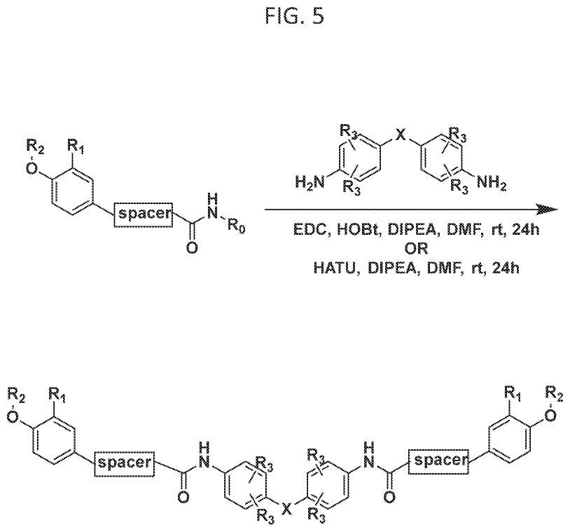

The synthesis of the 15 final compounds required an emphasis on speed, efficiency, and simplicity. As shown in FIG. 5, a small set of “head” groups was used and permuted in a wide variety of combinations, lowering cost and overall complexity. Likewise, the retrosynthetic design of pathways shown in FIGS. 4 and 5 emphasized rapid, simple synthetic steps with a common set of inexpensive reactants. At its most complex, the synthesis of particular Class I reactants required asymmetrical addition of two separate “heads,” requiring an intermediate deprotection step. However, most compounds were simple 1-2 step reactions, reducing the time and effort required to synthesize the set.

Purification efforts are ongoing in those compounds determined to be >97% pure via HPLC. These efforts primarily involve column chromatographic methods or simple separation to remove impurities, byproducts, and leftover starting materials. Once ¹H/¹³C NMR and HPLC have fully characterized a compound, it can be sent for further biological study. These studies include cell-based assays and in vitro/in vivo testing to quantify a given analog's specificity, toxicity, and potency when targeting ARID4B. Biological characterization is ongoing, and its exact methods and results are beyond the scope of this paper.

The overarching goal of this project is to synthesize potent inhibitors of the ARID4B pathway. More broadly, the iterative in silico-aided nature of this project's small molecule design process can be expanded upon and applied to other fields. Furthermore, such a process allowed for the rapid creation of novel, first-in-class small molecule inhibitors such as ARD150 in the absence of a crystal structure of the target protein.

Research into this family of compounds is an ongoing and collaborative effort. As the synthesis and characterization of new analogs are completed, they are characterized biologically in varied mouse models. The theoretical Structure-in-silico Activity Relationship (SiAR) and biological characterization results can be combined to determine which structural elements contribute most heavily towards potency. This cycle can be repeatedly iterated to develop increasingly potent and effective analogs of ARD150.

These products could be used as lead compounds to develop drugs that target the ARID4B pathway. An iterative SiAR-synthesis cycle can be used in future stages of development, as well, to optimize further aspects of these analogs for optimal pharmacokinetics. Successful creation of a potent and effective small molecule inhibitor for ARID4B will be vital in treating certain aggressive breast cancers and may find applications as a therapeutic intervention for related diseases.

### Compound Studies

Four distinct structural scaffolds (ARD150, ARD153, RWR-10, and RWR-18) that target the chromodomain of ARID4B with strong interactions with amino acids in their respective binding pockets (FIG. 14D) were synthesized. Based on predicted ADMET and potential for chemical diversification, ARD150 and RWR-18 were then focused on.

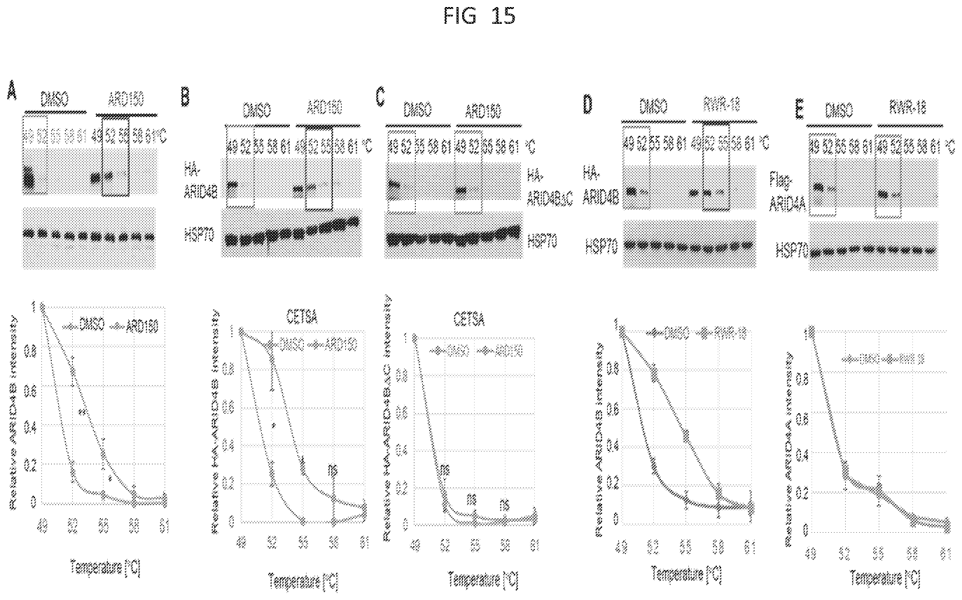

Using CETSA (cellular thermal shift assay) to examine whether ARD150 or RWR-18 binds to ARID4B, MCF7 cells treated or not with ARD150 or RWR-18 for 2 h were collected, followed by heating at 46, 49, 52, 55, and 58° C. to denature and precipitate ARID4B protein, cell lysis, removal of cell debris and aggregates by centrifugation, and finally detection of remaining thermostable ARID4B protein by western blot. The ARID4B aggregation temperatures without ARD150 were determined to be 49-52° C., but shifted to 52-55° C. after treatment with ARD150 and even more so with RWR-18 (FIG. 15A-D). To determine whether ARD150 and RWR-18 targets the chromodomain of ARID4B, CESTA was performed using chromodomain deletion mutant (ARID4BAC). HEK-293T cells expressing HA-ARID4B or HA-ARID4BAC were treated or not with ARD150 (10 μM) for 2 h and processed for CESTA. Similar to endogenous ARID4B, treatment with ARD150 resulted in a shift of aggregation temperatures of HA-ARID4B (FIG. 15B), whereas no shift was observed for HA-ARID4BAC (FIG. 15C). Similar results were obtained for RWR-18 (data not shown). Importantly, treatment of RWR-18 resulted in a shift of aggregation temperatures of HA-ARID4B (FIG. 9D), but not Flag-ARID4A (FIG. 15E).

Isothermal titration calorimetry (ITC) was used to confirm the binding of ARD150 and its derivative RWR18 (derived from ARD150 with slight structural modifications) to ARID4B chromodomain and to determine the K_{d}, reaction stoichiometry (n), enthalpy (ΔH) and entropy (ΔS). The results showed the K_{d} of ARD150 and RWR18 for ARID4B chromodomain are 3.5 μM and 1 μM, respectively (FIGS. 10, n, ΔH and ΔS were shown in the insets). This highlights to power of rational design to tune the target engagement and affinity of ligands.

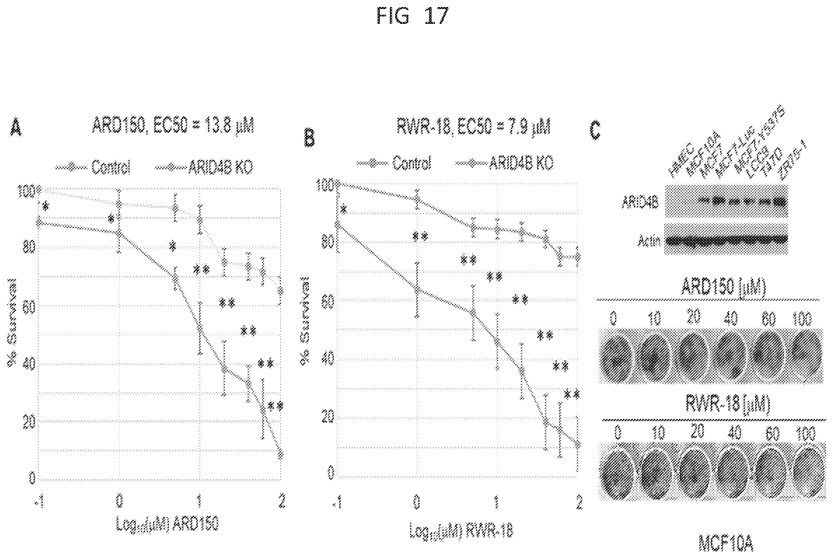

To determine the EC50 of ARD150 and two of its derivatives in breast cancer, MCF7, ZR75-1 (wild-type ERα+), and MCF7 Y537S (constitutively activate ERα mutant) and their ARID4B KO counterparts were seeded in 24 well plate and treated with the various concentrations (0, 0.1, 1, 5, 10, 20, 40, 60, and 100 μM) of ARD150 and RWR-18 for 14 days. The results showed that ARID4B inhibitors inhibited proliferation of MCF7 control cells in a dose-dependent manner as determined by MTT assays. The EC50 of ARD150 and RWR-18 for MCF7 are 13.8 and 7.9 μM, respectively (FIG. 17). EC50 of ARD150 and RWR18 for T47D and MCF7 Y537S were comparable to that of MCF7 (data not shown). Knockout of ARID4B renders cells less sensitive to these compounds. These compounds had little or no effects on the non-tumorigenic MCF10A, which expressed substantially lower ARID4B compared to cancer cells (FIG. 17C), suggesting that the inhibitory effects of these compounds are by targeting ARID4B. Taken together, the data demonstrates selectivity of these candidates for ARID4B, noting that the cells used are ARID4A competent.

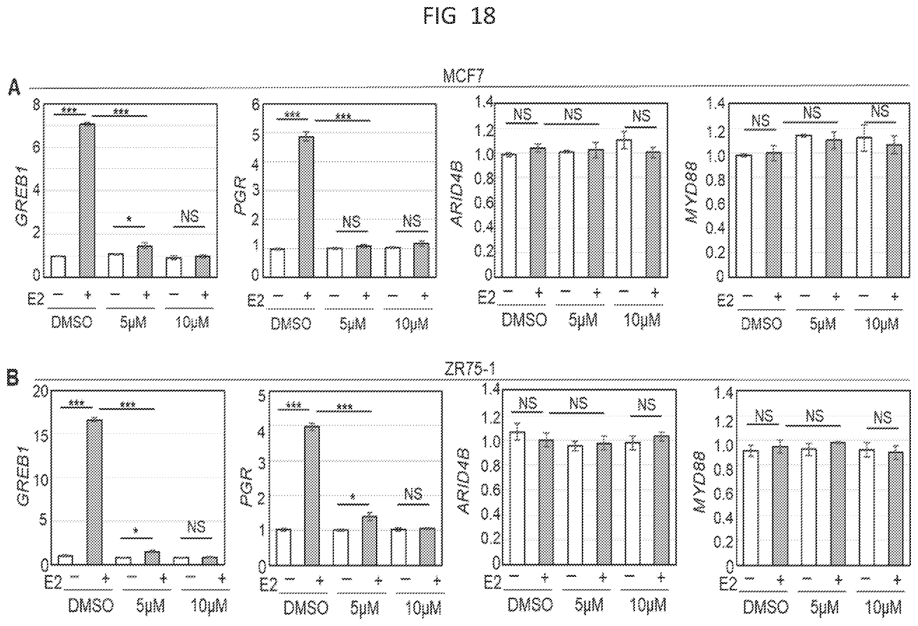

Consistent with knockout of ARID4B inhibited E2-induced and constitutive expression of ERα target genes, pre-treatment with RWR-18 (5 and 10 μM) abolished the induction of PGR and GREB1 genes by E2 in MCF7 and ZR75-1 cells (FIG. 18). The levels of ARID4B or MYD88 mRNA were not affected, indicating that suppression of PGR and GREB1 is not due to global suppression of transcription but a specific effect on ERα signaling.

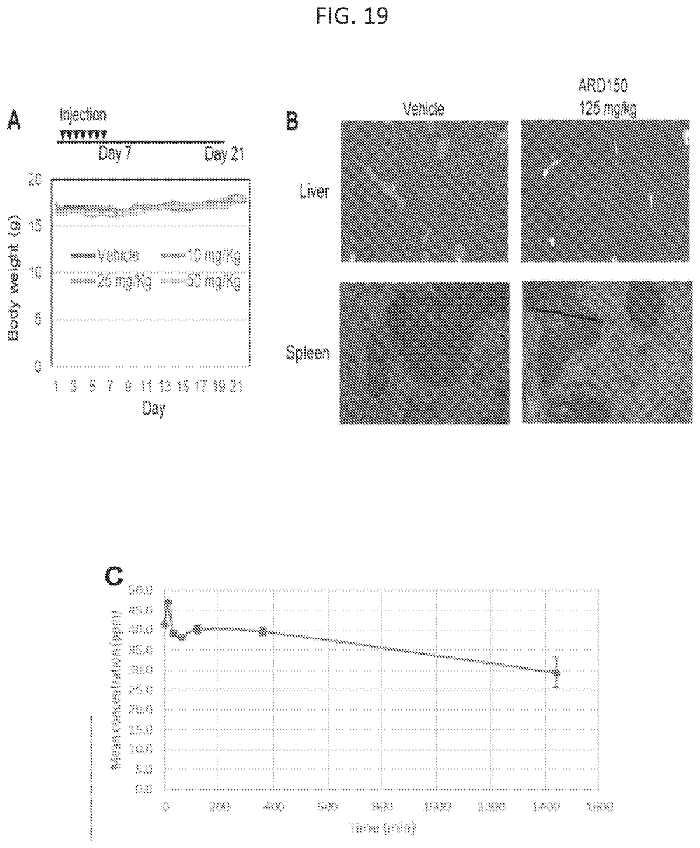

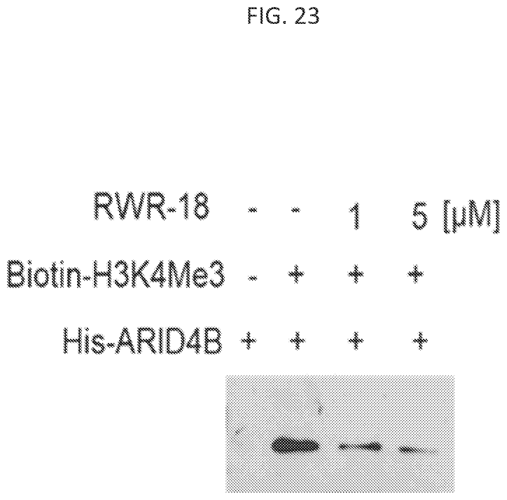

Since RWR-18 is more potent and has lower Kd than ARD150. The potential toxicity of RWR-18 was tested using 9-week-old Balb/c female mice. The mice were injected (i.p.) with vehicle or 10 mg/kg, 25 mg/kg, and 50 mg/kg of RWR-18 (daily for 7 days, 3 mice/group). The body weight was measured daily for 21 days. The liver, spleen, heart, lung, and kidney were collected for macroscopic and histological examination. The results showed that control mice and mice treated with RWR-18 at doses tested exhibited similar body weight gain (FIG. 19A). Macroscopic and histological examination did not reveal any toxicity and the survival rate was 100% (FIG. 19B and not shown). Preliminary plasma kinetic studies were performed. Following the incubation of the equivalent of 50 mg/kg of RWR-18 with blood plasma from mice at 10 min, 30 min, 1 h, 4 h, 16 h, and 24 h, we measured the plasma levels of RWR-18 by LC-MS. The results show RWR-18 remains intact over the 24 h period with ˜20% reduction in peak area, demonstrating good stability (FIG. 23C).

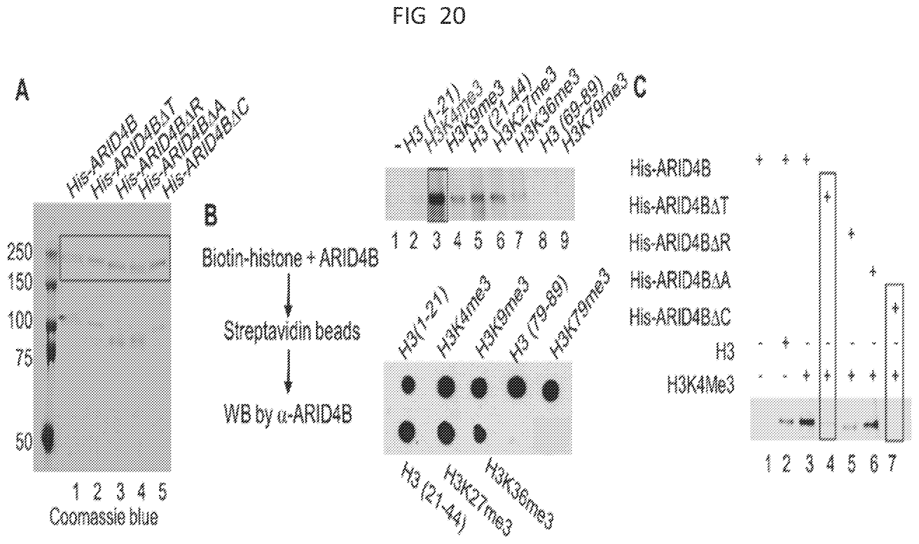

Histone peptide binding assays were used to determine that ARID4B binds to which methylated lysine. Full-length His-ARID4B and deletion mutants expressed in Sf9 were purified by Ni-NTA affinity resin (FIG. 20A). To determine which methylated lysine ARID4B binds, full-length His-ARID4B was incubated with biotinylated histone H3 peptides tri-methylated at the K4, K9, K27, K36 or K79. The corresponding unmethylated H3 peptides were used as controls (FIG. 8B). The results showed that ARID4B exhibited higher affinity for H3 peptide containing trimethylated K4 (H₃K4me3) compared to unmethylated H3 (FIG. 20B, compare lanes 3 to 2, 5, and 8), or other trimethylated histone peptides (FIG. 20B, compare lane 3 to 4, 6, 7, and 9). Next, the ARID4B deletion mutants were used to determine domain(s) of ARID4B responsible for binding to H₃K4me3. Deletion of Tudor and chromo domains impaired binding to H₃K4me3 (FIG. 20C, compare lanes 3, 4, and 7). Interestingly, deletion of RBB1NT domain, which had been suggested to function as a protein-protein interaction domain, also reduced binding to H₃K4me3 (FIG. 20C, compare lanes 3 and 5).

To provide a better understanding into the ARID4B binding landscape, ChIP-seq using ARID4B antibody was carried out in MCF7 cells. ChIP-seq analysis identified ARID4B enrichment on the promoters of ERα target genes, including PGR, GREB1, CCND1, SLC7A5 (FIG. 15A-B and not shown) that are involved in breast cancer. ER□ mostly binds to the enhancers and not promoters, expression of its target genes requires interaction of ER□-bound enhancers with target promoters (promoter-enhancer looping). Since ARID4B was recruited to the promoters that are far apart from the ER□-bound enhancers (FIGS. 15A-B), 3C-qPCR assays were performed to investigate the involvement of ARID4B in promoter-enhancer looping as a potential mechanism. 3C-qPCR results showed that looping of promoter-enhancer 1 and promoter-enhancer 2 of PGR gene were induced by E2 in control cells, and knockout of ARID4B abolished looping of promoter with enhancers 1 and 2 (FIG. 15C, compare green to red arrows). ARID4B was also required in the enhancer-promoter looping for GREB1 (not shown).

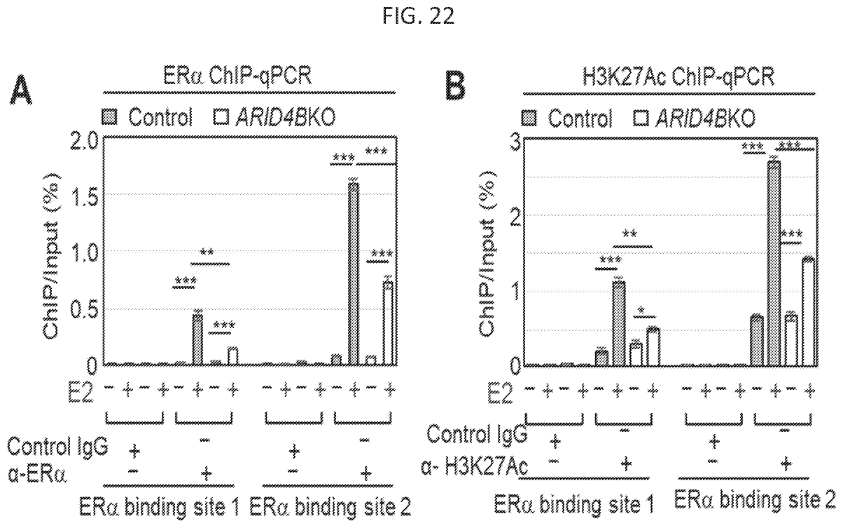

ChIP assays were performed to determine the recruitment of ERα to the enhancers in MCF7 cells with or without knockout of ARID4B. Consistently, ERα was recruited to the enhancers and not promoters of PGR and GREB1 upon treatment with E2, and knockout of ARID4B significantly reduced the E2-induced ERα recruitment to the enhancers of PGR and GREB1 (FIG. 22A, and not shown). Consistent with reduced ERα recruitment, E2-induced H3K27 acetylation (H3K27Ac), a transcription active marker, is reduced on the enhancers of PGR and GREB1 in ARID4B KO cells (FIG. 22B and not shown).

Since RWR-18 targets the chromodomain of ARID4B, which is important for binding to H3K4me3 (FIG. 20), it was tested whether RWR-18 interferes with binding of H3K4me3 using histone peptide binding assays as described above. As shown in FIG. 23, pre-treatment with RWR-18 inhibited binding of ARID4B to H3K4me3.

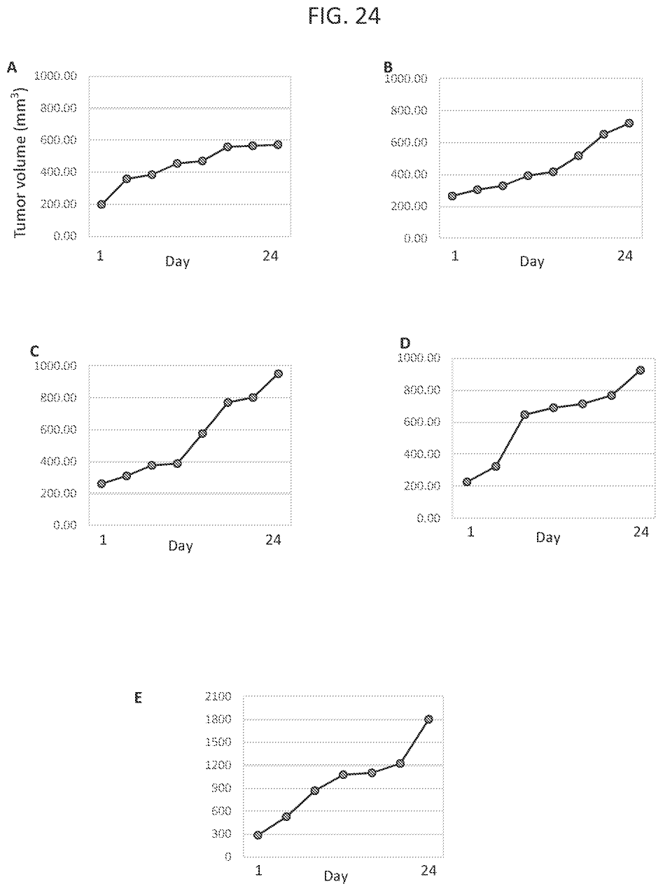

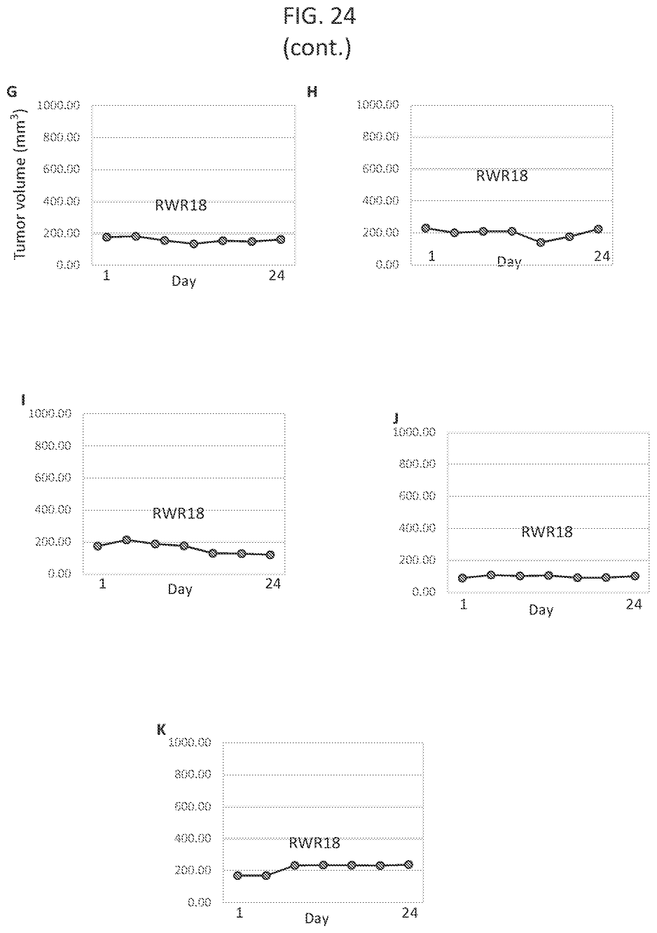

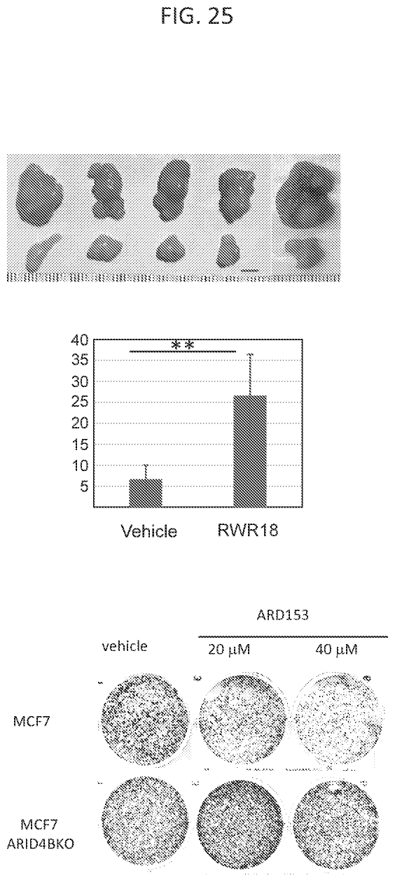

The effects of RWR18 on tumor growth was determined using MCF7 xenograft model. When tumor in SCID mice reached 150-200 mm³, mice were randomized to receive vehicle or RWR18 (25 mg/kg) once every two days for 24 days. The graphs showed tumor growth in mice receiving vehicle (FIG. 24A-E) or RWR18 (FIG. 24G-K). FIG. 25 shows are representative images of excised tumors from mice injected with vehicle (top) or RWR18 (bottom). FIG. 25 also shows that RWR18 increases the percentage of apoptotic cells. The bottom panel of FIG. 25 shows the effects observed with ARD153 in WT MCF7 cells and in ARID4B KO cells.

While particular aspects have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

It is appreciated that all reagents are obtainable by sources known in the art unless otherwise specified.

It is also to be understood that this disclosure is not limited to the specific aspects and methods described herein, as specific components and/or conditions may, of course, vary. Furthermore, the terminology used herein is used only for the purpose of describing particular aspects of the present disclosure and is not intended to be limiting in any way. It will be also understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein. Similarly, as used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference is made in detail to exemplary compositions, aspects and methods of the present disclosure, which constitute the best modes of practicing the disclosure presently known to the inventors. The Figures are not necessarily to scale. However, it is to be understood that the disclosed aspects are merely exemplary of the disclosure that may be embodied in various and alternative forms. Therefore, specific details disclosed herein are not to be interpreted as limiting, but merely as a representative basis for any aspect of the disclosure and/or as a representative basis for teaching one skilled in the art to variously employ the present disclosure.

Patents, publications, and applications mentioned in the specification are indicative of the levels of those skilled in the art to which the disclosure pertains. These patents, publications, and applications are incorporated herein by reference to the same extent as if each individual patent, publication, or application was specifically and individually incorporated herein by reference.

The foregoing description is illustrative of particular embodiments of the disclosure, but is not meant to be a limitation upon the practice thereof. The following claims, including all equivalents thereof, are intended to define the scope of the disclosure.

1. An ARD150 derivative compound comprising the structure as set forth in Formula (II), 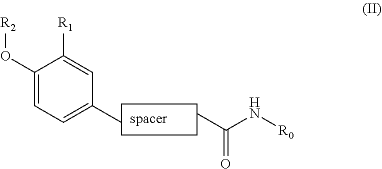
or a salt thereof
wherein the spacer is null or —OCH₂;
R₁ is null, H, or CH₃;
R₂ is H or CH₃; and,
R₀ is selected from: 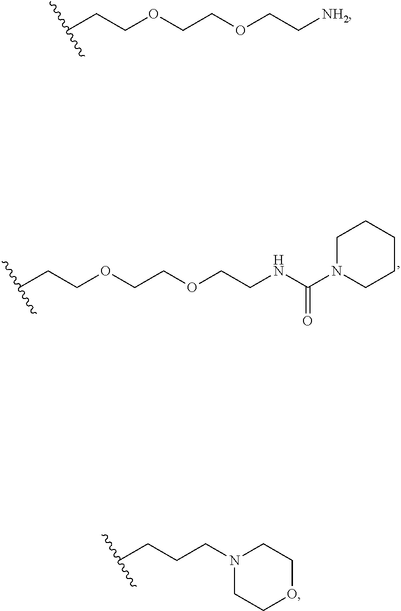 [SMILES: *CCOCCOCCN ; *CCOCCOCCNC(=O)N1CCCCC1 ; *CCCN1CCOCC1]
or a piperazine with the nitrogen of the depicted amide.

2. The ARD150 derivative compound of claim 1, wherein the compound is selected from 1a-1e: 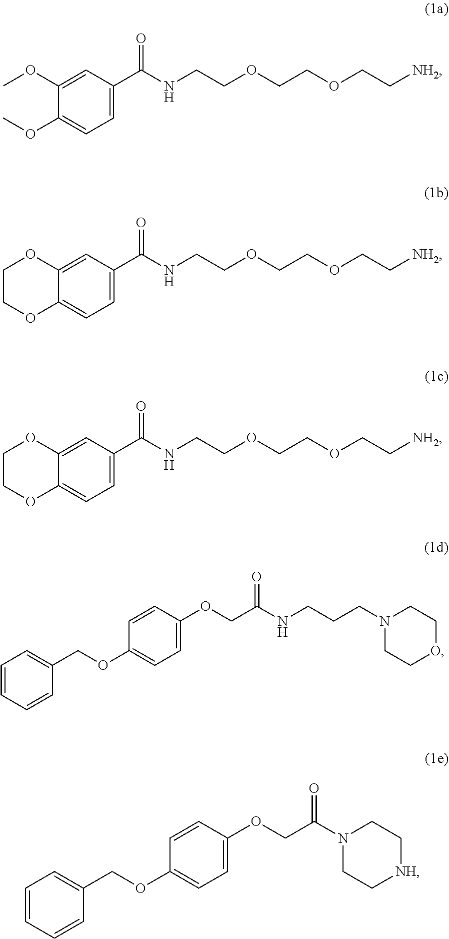 [SMILES: COc1ccc(C(=O)NCCOCCOCCN)cc1OC ; NCCOCCOCCNC(=O)c1ccc2c(c1)OCCO2 ; NCCOCCOCCNC(=O)c1ccc2c(c1)OCCO2 ; O=C(COc1ccc(OCc2ccccc2)cc1)NCCCN1CCOCC1 ; O=C(COc1ccc(OCc2ccccc2)cc1)N1CCNCC1]
or a salt thereof.

3. The ARD150 derivative compound of claim 1 or 2, wherein the compound comprises 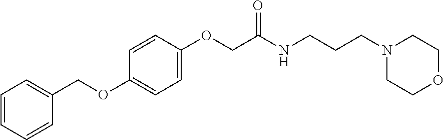 [SMILES: O=C(COc1ccc(OCc2ccccc2)cc1)NCCCN1CCOCC1]
or a salt thereof.

4. The ARD150 derivative compound of claim 1, wherein the composition of Formula II bookends a diamine with a basic structure as set forth in Formula III:

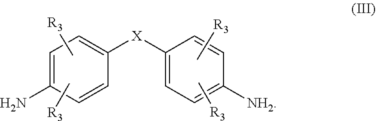

5. The ARD150 derivative compound of claim 4, wherein each R3 is independently null, C₆H₅OC₆H₅CH₂, or CH₃.

6. The ARD150 derivative compound of claim 4, wherein X is null, O, or S—S.

7. The ARD150 derivative compound of claim 4, wherein the diamine is selected from the following structures:

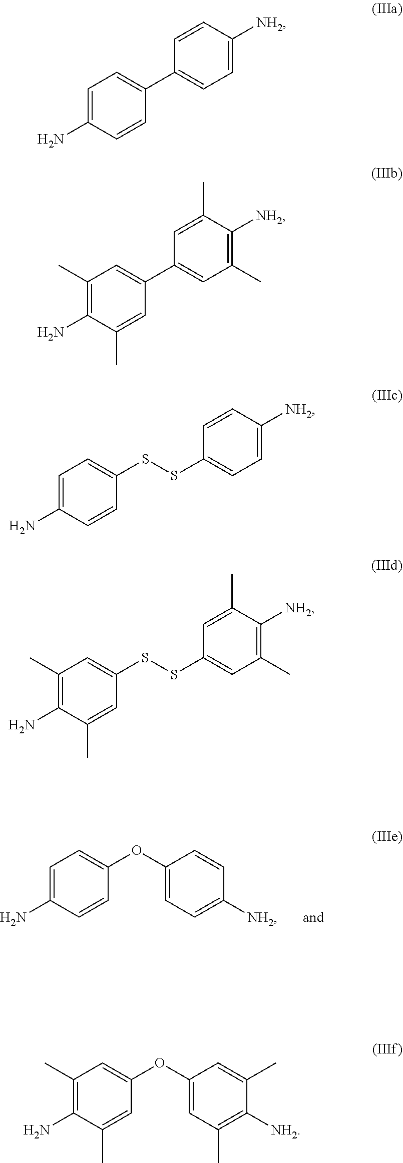 [SMILES: Nc1ccc(-c2ccc(N)cc2)cc1 ; Cc1cc(-c2cc(C)c(N)c(C)c2)cc(C)c1N ; Nc1ccc(SSc2ccc(N)cc2)cc1 ; Cc1cc(SSc2cc(C)c(N)c(C)c2)cc(C)c1N ; Nc1ccc(Oc2ccc(N)cc2)cc1 ; Cc1cc(Oc2cc(C)c(N)c(C)c2)cc(C)c1N]

8. The ARD150 derivative compound of claim 7, wherein the bookended or symmetrical compounds are set forth in Formula IV: 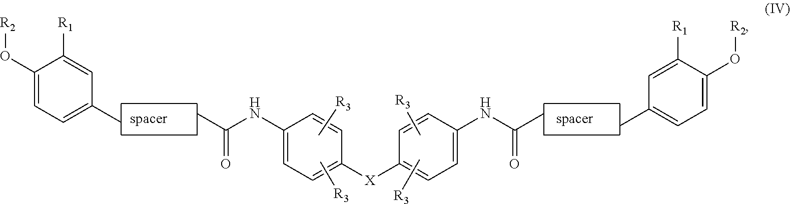
wherein each spacer is independently null or OCH₂;
each R₁ is independently null, H, or CH₃;
each R₂ is independently H or CH₃;
each R3 is independently null, C₆H₅OC₆H₅CH₂, or CH₃; and,
X is null, O, or S—S.

9. The ARD150 derivative compound of claim 7, wherein the compounds is selected from compounds 2a-2f as follow: 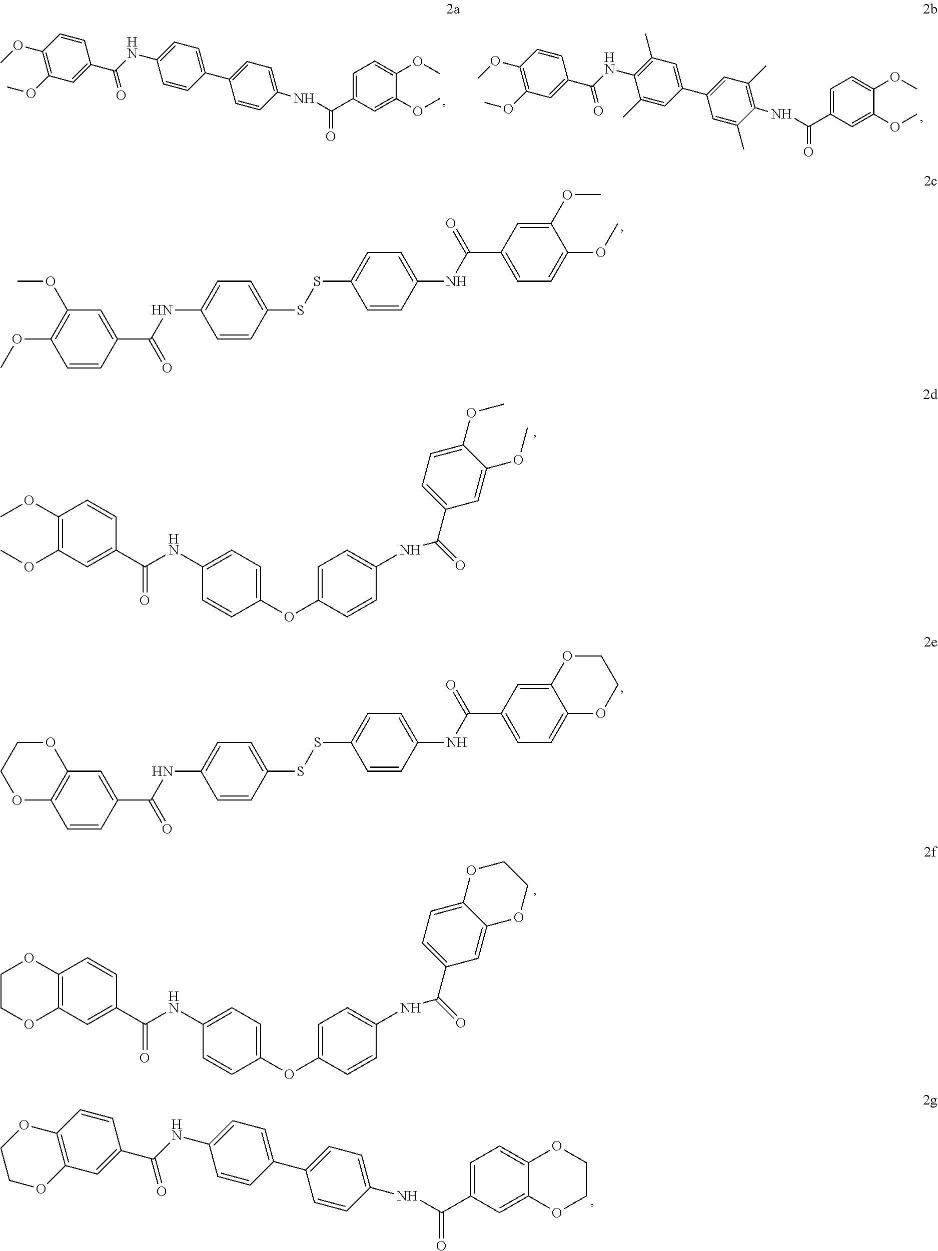 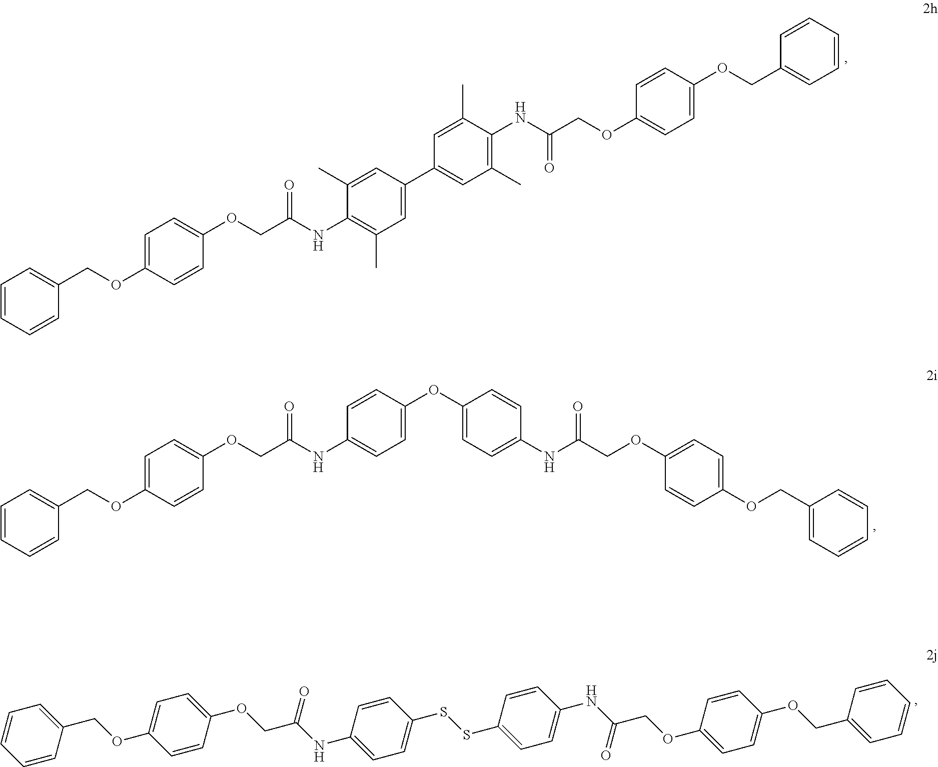 [SMILES: Cc1cc(-c2cc(C)c(NC(=O)COc3ccc(OCc4ccccc4)cc3)c(C)c2)cc(C)c1NC(=O)COc1ccc(OCc2ccccc2)cc1 ; O=C(COc1ccc(OCc2ccccc2)cc1)Nc1ccc(Oc2ccc(NC(=O)COc3ccc(OCc4ccccc4)cc3)cc2)cc1 ; O=C(COc1ccc(OCc2ccccc2)cc1)Nc1ccc(SSc2ccc(NC(=O)COc3ccc(OCc4ccccc4)cc3)cc2)cc1]
or a salt thereof.

10. The ARD150 derivative compound of claim 1, wherein the compounds comprises 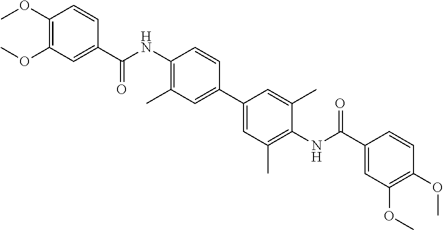
or a salt thereof.

11. The ARD150 derivative compound of claim 1, wherein the compound comprises 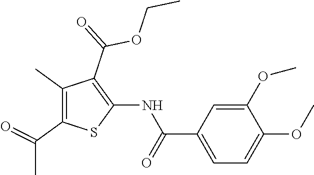 [SMILES: CCOC(=O)c1c(NC(=O)c2ccc(OC)c(OC)c2)sc(C(C)=O)c1C]
or a salt thereof.

12. A pharmecutical composition comprising the compouind of claim 1 and a pharmaceutically acceptable carrier.

13. The pharmacueitcla composiiton of claim 12, further compriusing an excipient.

14. A method for targeting AT-rich interactive domain 4B (ARID4B) in a cell, comprising administering the ARD150 derivative compound of claim 1 to a cell.

15. The method of claim 14, wherein the cell is a cancer cell.

16. The method of claim 14, wherein the cell is in vivo.

17. A method for treating cellular dysplasia in breast tissue of a subject comprising administering the compound of claim 1 to the subject.

18. The method of claim 17, wherein the compound is administered by a route selected from parenteral, topical, intravenous, oral, subcutaneous, sublingual, intraarterial, intradermal, transdermal, rectal, intracranial, intrathecal, intraperitoneal, intranasal; vaginally; intramuscular route or as inhalants.
